(19) United States
(12) Patent Application Publication
(10) Pub. No.: US 2021/0403567 A1
(43) Pub. Date: Dec. 30, 2021

(54) METHODS OF TREATING CERVICAL CANCER BY ADMINISTERING A PD-1 INHIBITOR

(71) Applicant: Regeneron Pharmaceuticals, Inc., Tarrytown, NY (US)

(72) Inventors: Matthew G. Fury, New York, NY (US); Israel Lowy, Dobbs Ferry, NY (US); Melissa Divya Mathias, Brooklyn, NY (US); Nazumi Alice Yama-Dang, Pleasantville, NY (US); Wen Fury, New York, NY (US)

(21) Appl. No.: 17/329,545

(22) Filed: May 25, 2021

Related U.S. Application Data
(60) Provisional application No. 63/185,881, filed on May 7, 2021.
(60) Provisional application No. 63/181,434, filed on Apr. 29, 2021.
(60) Provisional application No. 63/174,474, filed on Apr. 13, 2021.
(60) Provisional application No. 63/160,074, filed on Mar. 12, 2021.
(60) Provisional application No. 63/069,942, filed on Aug. 25, 2020.
(60) Provisional application No. 63/029,757, filed on May 26, 2020.

(51) Int. Cl. C07K 16/28; A61K 45/06; A61P 35/04

(52) U.S. Cl. CPC C07K 16/2818; A61K 2039/505; A61P 35/04; A61K 45/06

(57) ABSTRACT

The present disclosure provides methods for treating, reducing the severity of, or inhibiting the growth of a tumor or improving overall survival in a cervical cancer patient, wherein the method includes selecting a patient with cervical cancer in need thereof and administering to the patient a therapeutically effective amount of a programmed death 1 (PD-1) inhibitor (e.g., an anti-PD-1 antibody or antigen-binding fragment thereof such as cemiplimab or a bioequivalent thereof). In certain embodiments, the patient has recurrent or metastatic cervical cancer with disease progression on or after chemotherapy.

### FIELD

The present disclosure generally relates to methods of treating or inhibiting the growth of a tumor or improving overall survival of a cervical cancer patient, including selecting a patient with cervical cancer in need thereof and administering to the patient a therapeutically effective amount of a programmed death 1 (PD-1) inhibitor.

### BACKGROUND

Cervical cancer is the fourth most frequently diagnosed cancer and the fourth leading cause of cancer death in women worldwide, with approximately 570,000 cases per year and approximately 311,000 related deaths in 2018. (Bray et al., CA Cancer J Clin, 68 (2018) 394-424). Approximately 95% of cervical cancers stem from chronic infection with human papillomavirus (HPV). (Burk et al., Nature 543 (2017) 378-384). Approximately 80% of cervical cancers are classified as squamous cell carcinoma (arising from cells lining the bottom of the cervix) and the remainder are largely adenocarcinomas (arising from glandular cells in the upper cervix). Although vaccination against high risk strains of HPV is projected to gradually decrease the global incidence of cervical cancer in the next 15 years, the burden of this disease remains profound (Bray et al., Lancet Oncol, 2012; 13:790-801).

Cervical cancer is often curable when detected early and effectively managed, but treatment options are more limited in advanced stages. In the United States, approximately one-third of patients with cervical cancer will experience recurrent or metastatic disease and receive chemotherapy as first-line treatment. However, at least two-thirds of these patients will ultimately discontinue first-line chemotherapy due to disease progression, toxicity, or death.

For patients with locally advanced disease, curative intent therapy is definitive radiation with concurrent cisplatin. Patients with recurrent or metastatic disease are managed with chemotherapy in combination with bevacizumab when indicated. (Marth et al., Ann Oncol, 28 (2017) iv72-iv83). First-line treatment of these patients with the combination of cisplatin, paclitaxel, and bevacizumab is associated with increased overall survival (OS, 17.0 vs 13.3 months) and higher response rates (48% vs 36%) than the combination of cisplatin and paclitaxel alone. (Tewari et al., N Engl J Med, 370 (2014) 734-743). However, after progression on first line platinum-taxane based chemotherapy for recurrent or metastatic disease, there is no standard of care. Consequently, treatment options are limited for patients once their tumors progress on these regimens, and median survival is only approximately 7 months in the second-line or greater setting. (Marth et al., Ann Oncol 28 (2017) iv72-iv83; Lorusso et al., Ann Oncol 21 (2010) 61-66; Miller et al., Gynecol Oncol 110 (2008) 65-70). Pembrolizumab received accelerated approval from the Food and Drug Administration for the treatment of patients with recurrent or metastatic cervical cancer with disease progression on or after chemotherapy (whose tumors express programmed death-ligand 1 based on objective response rate and durability of responses. (Lorusso et al., Ann Oncol 21 (2010) 61-66). Yet, no agent has been shown to improve overall survival after first-line chemotherapy for metastatic cervical cancer. There is a need to develop treatment options for patients with recurrent or metastatic cervical carcinoma after progression on standard first-line platinum-taxane based chemotherapy with or without bevacizumab.

Expression of the immune checkpoint programmed death ligand-1 (PD-L1) is an immune-evasion strategy observed in both tumor cells and virally infected cells. PD-L1 expression has been detected in the majority of cervical squamous cell cancers using immunohistochemical analysis of tumor cells and the surrounding stroma (Heeren et al., Cancer Immunol Res 3 (2015) 48-58). Yet, there remains a need for safe and effective therapies for cervical cancer.

### SUMMARY

In one aspect, the disclosed technology relates to a method of treating or inhibiting the growth of a tumor or improving overall survival of a cervical cancer patient, including: selecting a patient with cervical cancer; and administering to the patient a therapeutically effective amount of a programmed death 1 (PD-1) inhibitor. In some embodiments, the cervical cancer is selected from the group consisting of squamous cell carcinoma, adenocarcinoma, and adenosquamous carcinoma. In some embodiments, the cervical cancer is squamous cell carcinoma of the cervix. In some embodiments, the cervical cancer is advanced, recurrent, persistent, and/or metastatic. In some embodiments, the cervical cancer is recurrent or metastatic. In some embodiments, the patient has disease progression on or after chemotherapy. In some embodiments, the patient has recurrent or metastatic cervical cancer with disease progression on or after chemotherapy. In certain embodiments, the patient has cervical cancer with squamous cell carcinoma histology. In certain embodiments, the patient has cervical cancer for which there is not a curative intent option (e.g., surgery or radiation therapy with or without chemotherapy). In some such embodiments, the patient is not a candidate for curative surgery or curative radiation. In some embodiments, the patient has received prior treatment such as chemotherapy (e.g., paclitaxel) or anti-VEGF therapy (e.g., bevacizumab). In some such embodiments, the patient is resistant or refractory to prior therapy. In some embodiments, the patient has received prior anti-cancer therapy, which was discontinued due to progression of disease and/or toxicity. In some embodiments, the prior anti-cancer therapy (e.g., chemotherapy or bevacizumab) is not appropriate for the patient with cervical cancer.

In some embodiments, the patient has received prior anti-cancer therapy. In some embodiments, the patient is resistant to, or the cervical cancer progressed after, prior treatment with an anti-cancer therapy. In some embodiments, the prior anti-cancer therapy comprises one or more of chemotherapy, surgery, radiation therapy, and/or anti-VEGF therapy. In some embodiments, the prior anti-cancer therapy comprises a platinum-based chemotherapy selected from pemetrexed, topotecan, irinotecan, gemcitabine, and vinorelbine. In some embodiments, the cervical cancer exhibits elevated expression of PD-L1. In some embodiments, the cervical cancer exhibits elevated expression of PD-L1 protein. In some embodiments, the cervical cancer exhibits elevated expression of PD-L1 mRNA. In some embodiments, the patient has tested positive for human papillomavirus (HPV). In some embodiments, the patient has tested negative for human papillomavirus (HPV).

In certain embodiments, the PD-1 inhibitor is administered as a monotherapy. In certain embodiments, the administration of the PD-1 inhibitor promotes tumor regression, reduces tumor cell load, reduces tumor burden, and/or prevents tumor recurrence in the patient. In some embodiments, the administration of the PD-1 inhibitor leads to at least one improvement selected from increase in overall survival, progression free survival, overall response rate, complete response, partial response, and stable disease, as compared to patients treated with chemotherapy. In some embodiments, the administration of the PD-1 inhibitor leads to increased overall survival as compared to patients treated with chemotherapy. In some embodiments, any of the above recited improvements occurs regardless of PD-L1 expression in the tumor.

In certain embodiments, the PD-1 inhibitor is administered in combination with a second therapeutic agent or therapy. In certain embodiments, the PD-1 inhibitor is selected from an anti-PD-1 antibody or antigen-binding fragment thereof, an anti-PD-L1 antibody or antigen-binding fragment thereof, and an anti-PD-L2 antibody or antigen-binding fragment thereof. In certain embodiments, the PD-1 inhibitor is selected from an anti-PD-1 antibody or antigen-binding fragment thereof.

In some embodiments, the PD-1 inhibitor is an anti-PD-1 antibody or antigen-binding fragment thereof that comprises a heavy chain variable region (HCVR) comprising three heavy chain complementarity determining regions (CDRs) (HCDR1, HCDR2 and HCDR3) and a light chain variable region (LCVR) comprising three light chain CDRs (LCDR1, LCDR2 and LCDR3), wherein: HCDR1 has an amino acid sequence of SEQ ID NO: 3; HCDR2 has an amino acid sequence of SEQ ID NO: 4; HCDR3 has an amino acid sequence of SEQ ID NO: 5; LCDR1 has an amino acid sequence of SEQ ID NO: 6; LCDR2 has an amino acid sequence of SEQ ID NO: 7; and LCDR3 has an amino acid sequence of SEQ ID NO: 8. In some embodiments, the HCVR comprises an amino acid sequence of SEQ ID NO: 1. In some embodiments, the LCVR comprises an amino acid sequence of SEQ ID NO: 2. In some embodiments, the anti-PD-1 antibody or antigen-binding fragment thereof comprises an HCVR/LCVR amino acid sequence pair of SEQ ID NOs: 1/2. In some embodiments, the anti-PD-1 antibody comprises a heavy chain and a light chain, wherein the heavy chain has an amino acid sequence of SEQ ID NO: 9. In some embodiments, the anti-PD-1 antibody comprises a heavy chain and a light chain, wherein the light chain has an amino acid sequence of SEQ ID NO: 10. In some embodiments, the anti-PD-1 antibody comprises a heavy chain and a light chain, wherein the heavy chain has an amino acid sequence of SEQ ID NO: 9 and the light chain has an amino acid sequence of SEQ ID NO: 10. In some embodiments, the PD-1 inhibitor is an anti-PD-1 antibody or antigen-binding fragment thereof comprising a HCVR with 90%, 95%, 97%, or 98% sequence identity to SEQ ID NO: 1. In some embodiments, the PD-1 inhibitor is an anti-PD-1 antibody or antigen-binding fragment thereof comprising a LCVR with 90%, 95%, 97%, or 98% sequence identity to SEQ ID NO: 2. In some embodiments, the PD-1 inhibitor is an anti-PD-1 antibody or antigen-binding fragment thereof comprising a HCVR with 90%, 95%, 97%, or 98% sequence identity to SEQ ID NO: 1, and a LCVR with 90%, 95%, 97%, or 98% sequence identity to SEQ ID NO: 2. In some embodiments, the PD-1 inhibitor is cemiplimab or a bioequivalent thereof.

In some embodiments, the PD-1 inhibitor is an anti-PD-1 antibody selected from the group consisting of cemiplimab, nivolumab, pembrolizumab, pidilizumab, MEDI0608, BI 754091, PF-06801591, spartalizumab, camrelizumab, JNJ-63723283, and MCLA-134. In other embodiments, the PD-1 inhibitor is an anti-PD-L1 antibody selected from the group consisting of REGN3504, avelumab, atezolizumab, durvalumab, MDX-1105, LY3300054, FAZ053, STI-1014, CX-072, KN035, and CK-301. In some embodiments, the PD-1 inhibitor is administered at a dose of 5 mg to 1500 mg. In some embodiments, the PD-1 inhibitor is administered at a dose of 200 mg, 250 mg, 350 mg, 400 mg, 500 mg, 600 mg, 700 mg, 750 mg, 800 mg, 1000 mg, 1050 mg, or 1200 mg. In some embodiments, the PD-1 inhibitor is administered at a dose of 1 mg/kg to 20 mg/kg of the patient's body weight. In some embodiments, the PD-1 inhibitor is administered at a dose of 1 mg/kg, 3 mg/kg or 10 mg/kg of the patient's body weight. In some embodiments, the PD-1 inhibitor is administered as one or more doses, wherein each dose is administered every week, two weeks, three weeks, four weeks, five weeks or six weeks. In certain embodiments, the PD-1 inhibitor is administered intravenously, subcutaneously, or intraperitoneally.

In another aspect, the disclosed technology relates to a programmed death 1 (PD-1) inhibitor for use in a method of treating or inhibiting the growth of a tumor or improving overall survival of a cervical cancer patient, the method including: (a) selecting a patient with cervical cancer; and (b) administering to the patient a therapeutically effective amount of a PD-1 inhibitor. In some embodiments, the cervical cancer is recurrent or metastatic cervical cancer with disease progression on or after chemotherapy or for whom chemotherapy is not appropriate.

In another aspect, the disclosed technology relates to a kit including a programmed death 1 (PD-1) inhibitor in combination with written instructions for use of a therapeutically effective amount of the PD-1 inhibitor for treating or inhibiting the growth of a tumor or improving overall survival of a patient with cervical cancer.

### BRIEF DESCRIPTION OF THE FIGURES

FIG. 1 is a box plot of PD-L1 mRNA expression in The Cancer Genome Atlas (TCGA) cervical cancers by histology, as described in Example 1.

FIG. 2 is a schematic diagram of the study described in Example 2.

FIG. 3 is a graph showing the mean change from baseline in Global Health Status/Quality of Life scale, mixed model repeated measure estimates in the overall population of patients described in Example 4.

FIG. 4 is a Kaplan-Meier curve of overall survival in the overall population (full analysis set) of patients with squamous cell carcinoma (SCC) and non-SCC histology in the study described in Example 4.

FIG. 5 is a Kaplan-Meier curve of overall survival in SCC patients (full analysis set) of the patients with SCC histology in the study described in Example 4.

FIG. 6 is a Kaplan-Meier curve of overall survival in adenocarcinoma patients (full analysis set) of the patients with adenocarcinoma/adenosquamous histology in the study described in Example 4.

FIG. 7 is a Kaplan-Meier curve of progression free survival in the overall population (full analysis set) of patients with squamous cell carcinoma (SCC) and non-SCC histology in the study described in Example 4.

FIG. 8 is a Kaplan-Meier curve of progression free survival in SCC patients (full analysis set) of the patients with SCC histology in the study described in Example 4.

FIG. 9 is a Kaplan-Meier curve of progression free survival in adenocarcinoma patients (full analysis set) of the patients with adenocarcinoma/adenosquamous histology in the study described in Example 4.

### DETAILED DESCRIPTION

It is to be understood that the present disclosure is not limited to the particular methods and experimental conditions described, as such methods and conditions may vary. It is also to be understood that the terminology used herein is for the purpose of describing particular embodiments only, and is not intended to be limiting, and that the scope of the present disclosure will be limited only by the appended claims. Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, preferred methods and materials are now described. All publications mentioned herein are hereby incorporated by reference in their entirety unless otherwise stated.

### Methods of Treating or Inhibiting Growth of Cervical Cancer

The present disclosure includes methods for treating or inhibiting the growth of a tumor or improving overall survival of a cervical cancer patient, comprising selecting a patient with cervical cancer and administering to the patient in need thereof an antibody or antigen-binding fragment thereof that specifically binds PD-1, PD-L1, and/or PD-L2, or any other “PD-1 inhibitor” as described herein. In the present disclosure, references to particular anti-PD-1 antibodies are provided to illustrate a representative PD-1 inhibitor, and do not limit the scope of the disclosure.

In some embodiments, the disclosed methods provide a surprisingly effective immunotherapy that improves overall survival of cervical cancer patients, as compared to cervical cancer patients treated with chemotherapy. In some embodiments, administering to a cervical cancer patient a therapeutically effective amount of an anti-PD-1 antibody (e.g., cemiplimab or a bioequivalent thereof) leads to improved overall survival, as compared to a cervical cancer patient treated with chemotherapy or another anti-PD-1 antibody (e.g., pembrolizumab or nivolumab). In some embodiments, administering to a cervical cancer patient a therapeutically effective amount of an anti-PD-1 antibody (e.g., cemiplimab or a bioequivalent thereof) provides an improved safety profile and leads to a lower incidence of adverse events as compared to a cervical cancer patient treated with chemotherapy. In some embodiments, administering to a cervical cancer patient a therapeutically effective amount of an anti-PD-1 antibody (e.g., cemiplimab or a bioequivalent thereof) leads to an improved overall mean change from baseline in Quality of Life of the patient as compared to a cervical cancer patient treated with chemotherapy.

The methods of the present disclosure provide an unexpectedly effective treatment across cervical cancer patient populations, including patients with squamous cell carcinoma, adenocarcinoma, and adenosquamous carcinoma, regardless of PD-L1 expression in the tumor. The disclosed methods thus provide a significant advantage in being effective against not only squamous forms of cervical cancer, but also adenocarcinoma which is particularly difficult to treat. The methods of the present disclosure also provide an unexpectedly effective second line therapy as a treatment for cervical cancer patients who were previously treated with and/or whose cervical cancer progressed on chemotherapy (e.g., platinum-based chemotherapy, such as pemetrexed, topotecan, irinotecan, gemcitabine, or vinorelbine), or for whom chemotherapy is not appropriate.

In some embodiments, the methods of the present disclosure do not require the cervical cancer patient to undergo PD-L1 testing prior to treatment with a PD-1 inhibitor, such as an anti-PD-1 antibody or antigen-binding fragment thereof (e.g., cemiplimab or a bioequivalent thereof). In this aspect, the disclosed methods include administering a therapeutically effective amount of the PD-1 inhibitor to a cervical cancer patient who does not and need not exhibit a threshold expression of PD-L1. In other embodiments, the cervical cancer patient expresses about 1%, about 2%, about 5%, about 10%, about 20%, about 30%, about 40%, about 50% or more PD-L1 expression in cancer tissue and/or tumor-infiltrating immune cells.

As used herein, the terms “treating”, “treat”, or the like, mean to alleviate or reduce the severity of at least one symptom or indication, to eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, to eliminate the need for radiation or surgery, and/or to increase duration of survival of the subject. In many embodiments, the terms “tumor”, “lesion,” “tumor lesion,” “cancer,” and “malignancy” are used interchangeably and refer to one or more cancerous growths.

In some embodiments, the cervical cancer is recurrent, persistent, and/or metastatic cervical cancer. In some embodiments, the cervical cancer is advanced cervical cancer. In some embodiments, the cervical cancer is squamous cell carcinoma (SCC) of the cervix. In some embodiments, the cervical cancer is adenocarcinoma. In some embodiments, the cervical cancer is adenosquamous carcinoma. In some embodiments, the patient has cervical cancer for which there is not a curative intent option (e.g., surgery or radiation therapy with or without chemotherapy). In some embodiments, the patient with cervical cancer shows an elevated level of PD-L1 expression in tumor tissue, wherein the tumor tissue comprises tumor cells and tumor-infiltrating immune cells.

As used herein, the term “recurrent” refers to a frequent or repeated diagnosis of cervical cancer in a patient or a frequent or repeated occurrence of individual tumors, such as primary tumors and/or new tumors that may represent recurrence of a prior tumor. In certain embodiments, administration of the PD-1 inhibitor inhibits the recurrence of a cervical cancer tumor in the patient.

As used herein, the expression “a subject in need thereof” means a human or non-human mammal that exhibits one or more symptoms or indications of cervical cancer, and/or who has been diagnosed with cervical cancer, and who needs treatment for the same. In many embodiments, the terms “subject” and “patient” are used interchangeably. The expression includes subjects with primary, established, recurrent or metastatic tumors (advanced malignancies). In specific embodiments, the expression includes human subjects that have and/or need treatment for recurrent and/or metastatic cervical cancer. The expression also includes subjects with persistent cervical cancer disease (disease for which there is no complete resolution after chemoradiation). In certain embodiments, the expression includes patients with cervical cancer that is resistant to or refractory to or is inadequately controlled by prior therapy (e.g., surgery or chemotherapy such as carboplatin or docetaxel). In certain embodiments, the expression includes subjects with cervical cancer who are not candidates for curative surgery or curative radiation, or for whom conventional anti-cancer therapy is inadvisable, for example, due to toxic side effects. In certain embodiments, the expression includes patients with cervical cancer that have received prior chemotherapy or any other anti-cancer therapy, progressed on such treatment, or been unsuitable (or not appropriate) for such treatment (e.g., patients that have received prior paclitaxel and/or prior bevacizumab, or that have been deemed unsuitable for such treatment). In certain embodiments, the expression includes patients with cervical cancer that have been treated with platinum, paclitaxel, and/or bevacizumab and had disease progression. In one embodiment, the expression includes patients with platinum-refractory cervical cancer.

In certain embodiments, the methods of the present disclosure may be used to treat patients with cervical cancer that show elevated levels of one or more cancer-associated biomarkers—e.g., programmed death ligand 1 (PD-L1), HPV oncogenes E6 or E7. In one embodiment, the methods of the present disclosure include administering a therapeutically effective amount of a PD-1 inhibitor (e.g., an anti-PD-1 antibody or antigen-binding fragment thereof) to a patient with an elevated level of PD-L1 in tumor tissue. In another embodiment, the methods are used in patients with cervical cancer that are selected on the basis of PD-L1 expression in cancer tissue. In certain embodiments, the methods of the present disclosure are used to treat patients with cervical cancer wherein the patients are selected on the basis of at least 1%, at least 2%, at least 5%, at least 10%, at least 20%, at least 30%, at least 40% or at least 50% PD-L1 expression in cancer tissue and/or tumor-infiltrating immune cells. Methods to determine PD-L1 expression in cancer tissue and/or immune cells are known in the art. In certain embodiments, the expression of PD-L1 in tumor tissue is determined by any assay known in the art, for example, by an ELISA assay or by an immunohistochemistry (IHC) assay, as described, e.g., in WO 2016124558, WO 2016191751, or US 20160305947. In certain embodiments, the expression of PD-L1 is determined by quantitating RNA expression, for example, by in situ hybridization or by RT-PCR. In certain embodiments, the expression of PD-L1 is determined by imaging with a labeled anti-PD-L1 antibody, for example, by immuno-positron emission tomography or iPET. See, e.g., The Oncologist, 12: 1379 (2007); Journal of Nuclear Medicine, 52(8): 1171 (2011); US 20180161464.

In some embodiments, the methods of the present disclosure may be used to treat patients with cervical cancer that test positive for HPV. In other embodiments, the methods of the present disclosure may be used to treat patients with cervical cancer that test negative for HPV.

In certain embodiments, the disclosed methods include administering a therapeutically effective amount of a PD-1 inhibitor (e.g., an anti-PD-1 antibody or antigen-binding fragment thereof) in combination with an anti-tumor therapy. Anti-tumor therapies include, but are not limited to, conventional anti-tumor therapies such as chemotherapy, radiation, surgery, or as elsewhere described herein.

The methods of the present disclosure, according to certain embodiments, include administering to a subject a therapeutically effective amount of a PD-1 inhibitor (e.g., an anti-PD-1 antibody or antigen-binding fragment thereof) in combination with a second therapeutic agent or therapy. The second therapeutic agent or therapy may be administered for increasing anti-tumor efficacy, for reducing toxic effects of one or more therapies and/or for reducing the dosage of one or more therapies. In various embodiments, the second therapeutic agent or therapy may include one or more of: radiation, surgery, a cancer vaccine, imiquimod, an anti-viral agent (e.g., cidofovir), photodynamic therapy, a programmed death ligand 1 (PD-L1) inhibitor (e.g., an anti-PD-L1 antibody), a lymphocyte activation gene 3 (LAG3) inhibitor (e.g., an anti-LAG3 antibody), a cytotoxic T-lymphocyte-associated protein 4 (CTLA-4) inhibitor (e.g., ipilimumab), a glucocorticoid-induced tumor necrosis factor receptor (GITR) agonist (e.g., an anti-GITR antibody), a T-cell immunoglobulin and mucin containing −3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an indoleamine-2,3-dioxygenase (IDO) inhibitor, a CD28 activator, a vascular endothelial growth factor (VEGF) antagonist [e.g., a “VEGF-Trap” such as aflibercept, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an angiopoietin-2 (Ang2) inhibitor, a transforming growth factor beta (TGFβ) inhibitor, an epidermal growth factor receptor (EGFR) inhibitor, an antibody to a tumor-specific antigen [e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9], a vaccine (e.g., Bacillus Calmette-Guerin), granulocyte-macrophage colony-stimulating factor, an oncolytic virus, a cytotoxin, a chemotherapeutic agent (e.g., pemetrexed, dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, topotecan, irinotecan, vinorelbine, and vincristine), an IL-6R inhibitor, an IL-4R inhibitor, an IL-10 inhibitor, a cytokine such as IL-2, IL-7, IL-12, IL-21, and IL-15, an antibody drug conjugate, an anti-inflammatory drug such as a corticosteroid, a non-steroidal anti-inflammatory drug (NSAID), cryotherapy, anti-HPV therapy, laser therapy, electrosurgical excision of cells with HPV, and a dietary supplement such as an antioxidant.

In certain embodiments, administering to a subject with cervical cancer a therapeutically effective amount of a PD-1 inhibitor (e.g., an anti-PD-1 antibody or antigen-binding fragment thereof) leads to increased inhibition of tumor growth—e.g., greater tumor regression in the treated subject. In certain embodiments, administering to a subject with cervical cancer a therapeutically effective amount of a PD-1 inhibitor (such as an anti-PD-1 antibody or antigen-binding fragment thereof) leads to increased tumor regression, tumor shrinkage and/or disappearance. In certain embodiments, the administration of a PD-1 inhibitor leads to delay in tumor growth and development, e.g., tumor growth may be delayed by about 3 days, more than 3 days, about 7 days, more than 7 days, more than 15 days, more than 1 month, more than 3 months, more than 6 months, more than 1 year, more than 2 years, or more than 3 years in the treated subject as compared to an untreated subject or a subject treated with platinum based chemotherapy or other SOC therapy such as those disclosed herein. In one embodiment, the increased inhibition of tumor growth occurs regardless of PD-L1 expression in the tumor.

In certain embodiments, administering to a subject with cervical cancer a therapeutically effective amount of a PD-1 inhibitor (e.g., an anti-PD-1 antibody or antigen-binding fragment thereof) prevents tumor recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months as compared to an untreated subject or a subject treated with platinum based chemotherapy or other ‘standard-of-care’ (SOC) therapy such as those disclosed herein. In one embodiment, the prevention of tumor recurrence and increase in the duration of survival occurs regardless of PD-L1 expression in the tumor.

In certain embodiments, administering to a subject with cervical cancer a therapeutically effective amount of a PD-1 inhibitor (e.g., an anti-PD-1 antibody or antigen-binding fragment thereof) leads to increased overall survival (OS) or progression-free survival (PFS) of the subject as compared to a subject administered with a SOC therapy. Non-limiting examples of standard of care therapy include platinum based chemotherapy (e.g., platinum-taxane based chemotherapy), antifolate (e.g., pemetrexed), topoisomerase 1 inhibitor (e.g., topotecan or irinotecan), nucleoside analogue (e.g., gemcitabine), vinca alkaloid (e.g., vinorelbine), surgery, radiation, and combinations thereof. In certain embodiments, the PFS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject administered with any one or more SOC therapies. In certain embodiments, the OS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a subject administered with any one or more SOC therapies (e.g., platinum based chemotherapy).

### PD-1 Inhibitors

The methods disclosed herein include administering a therapeutically effective amount of a PD-1 inhibitor. As used herein, a “PD-1 inhibitor” refers to any molecule capable of inhibiting, blocking, abrogating or interfering with the activity or expression of PD-1. In some embodiments, the PD-1 inhibitor can be an antibody, a small molecule compound, a nucleic acid, a polypeptide, or a functional fragment or variant thereof. Non-limiting examples of suitable PD-1 inhibitor antibodies include anti-PD-1 antibodies and antigen-binding fragments thereof, anti-PD-L1 antibodies and antigen-binding fragments thereof, and anti-PD-L2 antibodies and antigen-binding fragments thereof. Other non-limiting examples of suitable PD-1 inhibitors include RNAi molecules such as anti-PD-1 RNAi molecules, anti-PD-L1 RNAi, and an anti-PD-L2 RNAi, antisense molecules such as anti-PD-1 antisense RNA, anti-PD-L1 antisense RNA, and anti-PD-L2 antisense RNA, and dominant negative proteins such as a dominant negative PD-1 protein, a dominant negative PD-L1 protein, and a dominant negative PD-L2 protein. Some examples of the foregoing PD-1 inhibitors are described in e.g., U.S. Pat. No. 9,308,236, U.S. Ser. No. 10/011,656, and US 20170290808, the portions of which that identify PD-1 inhibitors are hereby incorporated by reference.

The term “antibody,” as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “VH”) and a heavy chain constant region (comprised of domains CH1, CH2 and CH3). Each light chain is comprised of a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In certain embodiments, the FRs of the antibody (or antigen binding fragment thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules.

As used herein, the terms “antigen-binding fragment” of an antibody, “antigen-binding portion” of an antibody, and the like, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) i V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}- C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The antibodies used in the methods disclosed herein may be human antibodies. As used herein, the term “human antibody” refers to antibodies having variable and constant regions derived from human germline immunoglobulin sequences. The human antibodies of the present disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods disclosed herein may be recombinant human antibodies. As used herein, the term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

### Anti-PD-1 Antibodies and Antigen-Binding Fragments Thereof

In some embodiments, PD-1 inhibitors used in the methods disclosed herein are antibodies or antigen-binding fragments thereof that specifically bind PD-1. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PD-1, as used in the context of the present disclosure, includes antibodies that bind PD-1 or a portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PD-1 may, however, have cross-reactivity to other antigens, such as PD-1 molecules from other (non-human) species.

According to certain exemplary embodiments, the anti-PD-1 antibody, or antigen-binding fragment thereof comprises a heavy chain variable region (HCVR), light chain variable region (LCVR), and/or complementarity determining regions (CDRs) comprising the amino acid sequences of any of the anti-PD-1 antibodies set forth in U.S. Pat. No. 9,987,500, which is hereby incorporated by reference in its entirety. In certain exemplary embodiments, the anti-PD-1 antibody or antigen-binding fragment thereof that can be used in the context of the present disclosure comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2. According to certain embodiments, the anti-PD-1 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8. In yet other embodiments, the anti-PD-1 antibody or antigen-binding fragment thereof comprises an HCVR comprising SEQ ID NO: 1 and an LCVR comprising SEQ ID NO: 2. In certain embodiments, the methods of the present disclosure comprise the use of an anti-PD-1 antibody, wherein the antibody comprises a heavy chain comprising the amino acid sequence of SEQ ID NO: 9. In some embodiments, the anti-PD-1 antibody comprises a light chain comprising the amino acid sequence of SEQ ID NO: 10. An exemplary antibody comprising a heavy chain variable region comprising the amino acid sequence of SEQ ID NO: 1 and a light chain variable region comprising the amino acid sequence of SEQ ID NO: 2 is the fully human anti-PD-1 antibody known as cemiplimab (also known as REGN2810; LIBTAYO®).

According to certain exemplary embodiments, the methods of the present disclosure comprise the use of cemiplimab or a bioequivalent thereof. As used herein, the term “bioequivalent” refers to anti-PD-1 antibodies or PD-1-binding proteins or fragments thereof that are pharmaceutical equivalents or pharmaceutical alternatives whose rate and/or extent of absorption do not show a significant difference with that of a reference antibody (e.g., cemiplimab) when administered at the same molar dose under similar experimental conditions, either single dose or multiple dose. In the context of the present disclosure, the term “bioequivalent” includes antigen-binding proteins that bind to PD-1 and do not have clinically meaningful differences with cemiplimab with respect to safety, purity and/or potency.

According to certain embodiments of the present disclosure, the anti-human PD-1, or antigen-binding fragment thereof, comprises a HCVR having 90%, 95%, 97% or 98% sequence identity to SEQ ID NO: 1.

According to certain embodiments of the present disclosure, the anti-human PD-1, or antigen-binding fragment thereof, comprises a LCVR having 90%, 95%, 97% or 98% sequence identity to SEQ ID NO: 2.

According to certain embodiments of the present disclosure, the anti-human PD-1, or antigen-binding fragment thereof, comprises a HCVR comprising an amino acid sequence of SEQ ID NO: 1 having no more than 5 amino acid substitutions. According to certain embodiments of the present disclosure, the anti-human PD-1, or antigen-binding fragment thereof, comprises a LCVR comprising an amino acid sequence of SEQ ID NO: 2 having no more than 2 amino acid substitutions.

Sequence identity may be measured by methods known in the art (e.g., GAP, BESTFIT, and BLAST).

The present disclosure also includes use of anti-PD-1 antibodies or antigen-binding fragments thereof in methods to treat cervical cancer, wherein the anti-PD-1 antibodies or antigen-binding fragments thereof comprise variants of any of the HCVR, LCVR and/or CDR amino acid sequences disclosed herein having one or more conservative amino acid substitutions. For example, the present disclosure includes use of anti-PD-1 antibodies or antigen-binding fragments thereof having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

Other anti-PD-1 antibodies or antigen-binding fragments thereof that can be used in the context of the methods of the present disclosure include, e.g., the antibodies referred to and known in the art as nivolumab, pembrolizumab, MEDI0608, pidilizumab, BI 754091, spartalizumab (also known as PDR001), camrelizumab (also known as SHR-1210), JNJ-63723283, MCLA-134, or any of the anti-PD-1 antibodies set forth in U.S. Pat. Nos. 6,808,710, 7,488,802, 8,008,449, 8,168,757, 8,354,509, 8,609,089, 8,686,119, 8,779,105, 8,900,587, and 9,987,500, and in patent publications WO 2006/121168, WO 2009/114335. The portions of all of the aforementioned publications that identify anti-PD-1 antibodies are hereby incorporated by reference.

The anti-PD-1 antibodies used in the context of the methods of the present disclosure may have pH-dependent binding characteristics. For example, an anti-PD-1 antibody for use in the methods of the present disclosure may exhibit reduced binding to PD-1 at acidic pH as compared to neutral pH. Alternatively, an anti-PD-1 antibody of the invention may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to PD-1 at acidic pH as compared to neutral pH” is expressed in terms of a ratio of the K_{D} value of the antibody binding to PD-1 at acidic pH to the K_{D} value of the antibody binding to PD-1 at neutral pH (or vice versa). For example, an antibody or antigen-binding fragment thereof may be regarded as exhibiting “reduced binding to PD-1 at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

### Anti-PD-L1 Antibodies and Antigen-Binding Fragments Thereof

In some embodiments, PD-1 inhibitors used in the methods disclosed herein are antibodies or antigen-binding fragments thereof that specifically bind PD-L1. For example, an antibody that “specifically binds” PD-L1, as used in the context of the present disclosure, includes antibodies that bind PD-L1 or a portion thereof with a K_{D} of about 1×10⁻⁸ M or less (e.g., a smaller K_{D} denotes a tighter binding). A “high affinity” anti-PD-L1 antibody refers to those mAbs having a binding affinity to PD-L1, expressed as K_{D} of at least 10⁻⁸ M, such as 10⁻⁹ M, 10⁻¹⁰ M, 10⁻¹¹ M, or 10⁻¹² M, as measured by surface plasmon resonance, e.g., BIACORE™ or solution-affinity ELISA. An isolated antibody that specifically binds human PD-L1 may, however, have cross-reactivity to other antigens, such as PD-L1 molecules from other (non-human) species.

According to certain exemplary embodiments, the anti-PD-L1 antibody or antigen-binding fragment thereof comprises a heavy chain variable region (HCVR), light chain variable region (LCVR), and/or complementarity determining regions (CDRs) comprising the amino acid sequences of any of the anti-PD-L1 antibodies set forth in U.S. Pat. No. 9,938,345, which is hereby incorporated by reference in its entirety. In certain exemplary embodiments, an anti-PD-L1 antibody or antigen-binding fragment thereof that can be used in the context of the present disclosure comprises the heavy chain complementarity determining regions (HCDRs) of a heavy chain variable region (HCVR) comprising SEQ ID NO: 11 and the light chain complementarity determining regions (LCDRs) of a light chain variable region (LCVR) comprising SEQ ID NO: 12. An exemplary anti-PD-L1 antibody comprising a HCVR of SEQ ID NO: 11 and a LCVR of SEQ ID NO: 12 is REGN3504.

According to certain embodiments of the present disclosure, the anti-human PD-L1 antibody, or antigen-binding fragment thereof, comprises a HCVR having 90%, 95%, 97% or 98% sequence identity to SEQ ID NO: 11. According to certain embodiments of the present disclosure, the anti-human PD-L1 antibody, or antigen-binding fragment thereof, comprises a LCVR having 90%, 95%, 97% or 98% sequence identity to SEQ ID NO: 12.

According to certain embodiments of the present disclosure, the anti-human PD-L1 antibody, or antigen-binding fragment thereof, comprises a HCVR comprising an amino acid sequence of SEQ ID NO: 11 having no more than 5 amino acid substitutions. According to certain embodiments of the present disclosure, the anti-human PD-L1 antibody, or antigen-binding fragment thereof, comprises a LCVR comprising an amino acid sequence of SEQ ID NO: 12 having no more than 2 amino acid substitutions.

Sequence identity may be measured by methods known in the art (e.g., GAP, BESTFIT, and BLAST).

The present disclosure also includes use of anti-PD-L1 antibodies in methods to treat cervical cancer, wherein the anti-PD-L1 antibodies comprise variants of any of the HCVR, LCVR and/or CDR amino acid sequences disclosed herein having one or more conservative amino acid substitutions. For example, the present disclosure includes use of anti-PD-L1 antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

Other anti-PD-L1 antibodies that can be used in the context of the methods of the present disclosure include, e.g., the antibodies referred to and known in the art as MDX-1105, atezolizumab (TECENTRIQ™), durvalumab (IMFINZI™), avelumab (BAVENCIO™) LY3300054, FAZ053, STI-1014, CX-072, KN035 (Zhang et al., Cell Discovery, 3, 170004 (March 2017)), CK-301 (Gorelik et al., American Association for Cancer Research Annual Meeting (AACR), 2016-04-04 Abstract 4606), or any of the other anti-PD-L1 antibodies set forth in patent publications U.S. Pat. Nos. 7,943,743, 8,217,149, 9,402,899, 9,624,298, 9,938,345, WO 2007/005874, WO 2010/077634, WO 2013/181452, WO 2013/181634, WO 2016/149201, WO 2017/034916, or EP3177649. The portions of all of the aforementioned publications that identify anti-PD-L1 antibodies are hereby incorporated by reference.

### Pharmaceutical Compositions and Administration

The present disclosure provides therapeutic pharmaceutical compositions comprising the PD-1 inhibitors disclosed herein. Such pharmaceutical compositions may be formulated with suitable pharmaceutically acceptable carriers, excipients, buffers, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al., “Compendium of excipients for parenteral formulations” PDA, J Pharm Sci Technol 52:238-311 (1998).

The dose of PD-1 inhibitor (e.g., anti-PD-1 antibody or antigen-binding fragment thereof) may vary depending upon the age and the size of a subject to be administered, target disease, conditions, route of administration, and the like. When a PD-1 inhibitor of the present disclosure is used for treating or inhibiting the growth of cervical cancer or improving overall survival of a cervical cancer patient, it may be advantageous to administer the PD-1 inhibitor at a single dose of about 0.1 to about 100 mg/kg body weight. Depending on the severity of the condition, the frequency and the duration of the treatment can be adjusted. In certain embodiments, the PD-1 inhibitor of the present disclosure can be administered as an initial dose of at least about 0.1 mg to about 1500 mg, about 1 to about 1000 mg, about 3 to about 800 mg, about 5 to about 500 mg, or about 10 to about 400 mg. In certain embodiments, the initial dose may be followed by administration of a second or a plurality of subsequent doses of the PD-1 inhibitor in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. The pharmaceutical composition can be also delivered in a vesicle, in particular a liposome (see, e.g., Langer (1990) Science 249:1527-1533).

The use of nanoparticles to deliver the PD-1 inhibitor of the present disclosure is also contemplated herein. Antibody-conjugated nanoparticles may be used both for therapeutic and diagnostic applications. Antibody-conjugated nanoparticles and methods of preparation and use are described in detail by Arruebo et al., 2009, “Antibody-conjugated nanoparticles for biomedical applications,” J. Nanomat., Vol. 2009, Article ID 439389, 24 pages. Nanoparticles may be developed and conjugated to antibodies contained in pharmaceutical compositions to target cells. Nanoparticles for drug delivery have also been described in, for example, U.S. Pat. No. 8,257,740, or U.S. Pat. No. 8,246,995.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracranial, intraperitoneal and intramuscular injections, drip infusions, etc. These injectable preparations may be prepared by methods publicly known.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 1500 mg per dosage form in a unit dose.

In certain embodiments, the present disclosure provides a pharmaceutical composition or formulation comprising a therapeutic amount of a PD-1 inhibitor (e.g., an anti-PD-1 antibody or antigen-binding fragment thereof) and a pharmaceutically acceptable carrier. Non-limiting examples of pharmaceutical compositions comprising an anti-PD-1 antibody that can be used in the context of the present disclosure are disclosed in US 2019/0040137.

The present disclosure also provides kits comprising a PD-1 inhibitor (e.g., an anti-PD-1 antibody or antigen-binding fragment thereof) for therapeutic uses as described herein. Kits typically include a label indicating the intended use of the contents of the kit and instructions for use. As used herein, the term “label” includes any writing, or recorded material supplied on, in or with the kit, or which otherwise accompanies the kit. Accordingly, this disclosure provides a kit for treating a patient afflicted with cervical cancer, the kit comprising: (a) a therapeutically effective dosage of a PD-1 inhibitor (e.g., an anti-PD-1 antibody or antigen-binding fragment thereof); and (b) instructions for using the PD-1 inhibitor in any of the methods disclosed herein.

### Administration Regimens

In certain embodiments, the methods disclosed herein include administering to the tumor of a subject in need thereof a therapeutically effective amount of a PD-1 inhibitor (e.g., an anti-PD-1 antibody or antigen-binding fragment thereof) in multiple doses, e.g., as part of a specific therapeutic dosing regimen. For example, the therapeutic dosing regimen may comprise administering one or more doses of a PD-1 inhibitor to the subject at a frequency of about once a day, once every two days, once every three days, once every four days, once every five days, once every six days, once a week, once every two weeks, once every three weeks, once every four weeks, once every five weeks, once every six weeks, once every eight weeks, once every twelve weeks, once a month, once every two months, once every three months, once every four months, twice a day, twice every two days, twice every three days, twice every four days, twice every five days, twice every six days, twice a week, twice every two weeks, twice every three weeks, twice every four weeks, twice every five weeks, twice every six weeks, twice every eight weeks, twice every twelve weeks, twice a month, twice every two months, twice every three months, twice every four months, three times a day, three times every two days, three times every three days, three times every four days, three times every five days, three times every six days, three times a week, three times every two weeks, three times every three weeks, three times every four weeks, three times every five weeks, three times every six weeks, three times every eight weeks, three times every twelve weeks, three times a month, three times every two months, three times every three months, three times every four months or less frequently or as needed so long as a therapeutic response is achieved. In one embodiment, one or more doses of a PD-1 inhibitor as set forth herein are administered once every three weeks. In one embodiment, one or more doses of a PD-1 inhibitor as set forth herein are administered once every six weeks. In one embodiment, one or more doses of a PD-1 inhibitor as set forth herein are administered once every three weeks, followed by one or more doses administered once every six weeks.

In some embodiments, one or more doses of a PD-1 inhibitor as set forth herein (e.g., an anti-PD-1 antibody or antigen-binding fragment thereof, such as cemiplimab or a bioequivalent thereof) are administered at a dose of 350 mg once every 3 weeks. In some embodiments, one or more doses of a PD-1 inhibitor as set forth herein are administered at a dose of 600 mg once every four weeks. In some embodiments, one or more doses of a PD-1 inhibitor as set forth herein are administered at a dose of 600 mg once every six weeks. In some embodiments, one or more doses of a PD-1 inhibitor as set forth herein are administered at a dose of 700 mg once every six weeks. In some embodiments, one or more doses of a PD-1 inhibitor as set forth herein are administered at a dose of 1050 mg once every six weeks.

In certain embodiments, the one or more doses are administered in at least one treatment cycle. The methods, according to this aspect, comprise administering to a subject in need thereof at least one treatment cycle comprising administration of 1, 2, 3, 4, 5, 6, 7, 8, 9, 10 or more doses of a PD-1 inhibitor (e.g., an anti-PD-1 antibody or antigen-binding fragment thereof). In one embodiment, a treatment cycle comprises 12 doses of a PD-1 inhibitor. In one embodiment, a treatment cycle comprises 24 doses of a PD-1 inhibitor.

### Dosage

The amount of PD-1 inhibitor (e.g., an anti-PD-1 antibody or antigen-binding fragment thereof) administered to a subject according to the methods disclosed herein is, generally, a therapeutically effective amount. As used herein, the term “therapeutically effective amount” means an amount of a PD-1 inhibitor that results in one or more of: (a) a reduction in the severity or duration of a symptom or an indication of cervical cancer—e.g., a tumor lesion; (b) inhibition of tumor growth, or an increase in tumor necrosis, tumor shrinkage and/or tumor disappearance; (c) delay in tumor growth and development; (d) inhibition of tumor metastasis; (e) prevention of recurrence of tumor growth; (f) increase in survival of a subject with a cancer; and/or (g) a reduction in the use or need for conventional anti-cancer therapy (e.g., elimination of need for surgery or reduced or eliminated use of chemotherapeutic or cytotoxic agents) as compared to an untreated subject or a subject treated with platinum based chemotherapy or other SOC therapy such as those disclosed herein.

In certain embodiments, a therapeutically effective amount of the PD-1 inhibitor (e.g., an anti-PD-1 antibody or antigen-binding fragment thereof, such as cemiplimab or a bioequivalent thereof) can be from about 0.05 mg to about 1500 mg, from about 1 mg to about 800 mg, from about 5 mg to about 600 mg, from about 10 mg to about 550 mg, from about 50 mg to about 400 mg, from about 75 mg to about 350 mg, or from about 100 mg to about 300 mg of the antibody. For example, in various embodiments, the amount of the PD-1 inhibitor is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, or about 1500 mg.

The amount of a PD-1 inhibitor contained within an individual dose may be expressed in terms of milligrams of antibody per kilogram of subject body weight (i.e., mg/kg). In certain embodiments, the PD-1 inhibitor used in the methods disclosed herein may be administered to a subject at a dose of about 0.0001 to about 100 mg/kg of subject body weight. In certain embodiments, an anti-PD-1 antibody may be administered at dose of about 0.1 mg/kg to about 20 mg/kg of a patient's body weight. In certain embodiments, the methods of the present disclosure comprise administration of a PD-1 inhibitor (e.g., an anti-PD-1 antibody or antigen-binding fragment thereof) at a dose of about 1 mg/kg to 3 mg/kg, 1 mg/kg to 5 mg/kg, 1 mg/kg to 10 mg/kg, 1 mg/kg, 3 mg/kg, 5 mg/kg, or 10 mg/kg of a patient's body weight.

In certain embodiments, an individual dose amount of a PD-1 inhibitor (e.g., an anti-PD-1 antibody or antigen-binding fragment thereof) administered to a patient may be less than a therapeutically effective amount, i.e., a subtherapeutic dose. For example, if the therapeutically effective amount of a PD-1 inhibitor comprises 3 mg/kg, a subtherapeutic dose comprises an amount less than 3 mg/kg, e.g., 2 mg/kg, 1.5 mg/kg, 1 mg/kg, 0.5 mg/kg or 0.3 mg/kg. As defined herein, a “subtherapeutic dose” refers to an amount of the PD-1 inhibitor that does not lead to a therapeutic effect by itself. However, in certain embodiments, multiple subtherapeutic doses of a PD-1 inhibitor are administered to collectively achieve a therapeutic effect in the subject.

In certain embodiments, each dose comprises 0.1-10 mg/kg (e.g., 0.3 mg/kg, 1 mg/kg, 3 mg/kg, or 10 mg/kg) of the subject's body weight. In certain other embodiments, each dose comprises 5-1500 mg of the PD-1 inhibitor (such as an anti-PD-1 antibody or antigen-binding fragment thereof), e.g., 5 mg, 10 mg, 15 mg, 20 mg, 25 mg, 30 mg, 40 mg, 45 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1550 mg, 1300 mg, 1350 mg, 1400 mg, 1450 mg, or 1500 mg of the PD-1 inhibitor.

In certain embodiments, the PD-1 inhibitor (e.g., an anti-PD-1 antibody or antigen-binding fragment thereof) is administered in combination with radiation therapy provided in one or more doses of 2-100 Gray (Gy). In certain embodiments, the radiation therapy comprises 2, 3, 4, 5, 6, 7, 8, 9, 10, 11, 12, 15, 20, 23, 25, 27, 30, 35, 40, or 45 Gy. In certain other embodiments, the radiation therapy comprises 50-100, 60-90, or 70-80 Gy. In certain embodiments, the radiation therapy is administered in fractions (hypofractionated radiation therapy). Hypofractionated radiation therapy (hfRT) refers to radiation therapy in which a radiation dose is comprised in 2 or more fractions. In various embodiments, each fraction comprises 2-20 Gy. For example, a radiation dose of 50 Gy may be split up into 10 fractions, each comprising 5 Gy. In certain embodiments, the 2 or more fractions are administered on consecutive or sequential days. In certain other embodiments, the 2 or more fractions are administered over a period of time comprising once in 2 days, once in 3 days, once in 4 days, once in 5 days, once in 6 days, once in 7 days, twice in 3 days, twice in one week, three times in one week, or a combination thereof.

### EXAMPLES

The following examples are put forth so as to provide those of ordinary skill in the art with a complete disclosure and description of how to make and use the methods and compositions of the present disclosure and are not intended to limit the scope of what the inventors regard as their invention. Likewise, the disclosure is not limited to any particular preferred embodiments described herein. Indeed, modifications and variations of the embodiments may be apparent to those skilled in the art upon reading this specification and can be made without departing from its spirit and scope. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Clinical Trial of Anti-PD-1 Antibody in Patients with Cervical Cancer

This study was a phase I, open-label, multicenter study of an anti-PD-1 antibody (cemiplimab) in patients with advanced solid tumors. Cemiplimab is a high-affinity, human, hinge-stabilized IgG4 monoclonal antibody to the PD-1 receptor that potently blocks the interactions of PD-1 with PD-L1 and PD-L2. Cemiplimab comprises a heavy chain having the amino acid sequence of SEQ ID NO: 9 and a light chain having the amino acid sequence of SEQ ID NO: 10; an HCVR/LCVR amino acid sequence pair comprising SEQ ID NOs: 1/2; and heavy and light chain CDR sequences comprising SEQ ID NOs: 3-8, as described herein. See also U.S. Pat. No. 9,987,500. The study included patients aged 18 years with histologically or cytologically confirmed recurrent or metastatic cervical cancer who were resistant to or intolerant of platinum and taxane doublet chemotherapy.

One group of patients [monotherapy cohort, for which hypofractionated radiation therapy (hfRT) was not planned] received cemiplimab 3 mg/kg intravenously (IV) every 2 weeks (Q2W) for 48 weeks. A second group of patients (combination therapy cohort, for which palliative radiotherapy was planned) received cemiplimab 3 mg/kg IV Q2W for up to 48 weeks plus hfRT (9 Gy×3 times over 1 week to a single lesion, starting 1 week after Day 1 of cemiplimab from Day 8 to Day 12 of a 14-day cycle). The combination therapy cohort enrolled patients for whom palliative hfRT was planned to a lesion that was causing some signs or symptoms. The radiated lesion was not followed as a target lesion for response assessments. Cemiplimab was supplied at a concentration of 25 or 50 mg/mL. Cemiplimab infusion was conducted for 30 minutes with a ±10 minute window.

Additional eligibility criteria included an Eastern Cooperative Oncology Group performance status score of 0 or 1, and adequate organ function. Patients were required to have at least one measurable lesion per Response Evaluation Criteria in Solid Tumors (RECIST) version 1.1. (Eisenhauer et al., Eur J Cancer 45 (2009) 228-47). In the combination therapy cohort, the measureable lesion(s) were in addition to the irradiated lesion.

Key exclusion criteria included ongoing or recent (within 5 years) autoimmune disease requiring systemic immunosuppression, prior treatment with an agent blocking the PD-1/PD-L1 pathway, a history of solid organ transplantation, concurrent cancer (unless indolent or non-life-threatening), or hematologic cancer.

Study objectives: One objective of this study was to characterize the safety and tolerability of cemiplimab as monotherapy or in combination with hfRT in patients with recurrent or metastatic cervical cancer. Another objective was to determine the anti-tumor activity of cemiplimab as monotherapy or in combination with hfRT in these patients.

Assessments: Tumor histology was assessed per local pathology reports of tumor samples obtained at any time prior to study enrollment. Severity of treatment-emergent adverse events (TEAEs) was graded according to the National Cancer Institute Common Terminology Criteria for Adverse Events (version 4.03). Extensive safety evaluations were performed during screening and on Day 1 of each subsequent treatment cycle throughout the study. Routine safety evaluations were performed at each cemiplimab dosing visit. Tumor response assessments were performed by investigators, per RECIST 1.1, at the end of each 8-week treatment cycle. In combination therapy cohorts, the lesions selected for hfRT were not included in RECIST 1.1 assessments.

Statistical analysis of clinical data: The safety summaries and analyses were performed on the safety analysis set. Data were summarized using descriptive statistics, along with two-sided 95% confidence interval (CI), by dose cohort. Continuous variables were summarized with mean, median, standard deviation, minimum, and maximum. Categorical variables were summarized with frequency and percentage. Kaplan-Meier analysis was performed for progression-free survival (PFS) and OS.

Analysis of PD-L1 protein and mRNA expression in independent cervical cancer samples: A total of 155 commercially available tumor blocks were evaluated for tumor-infiltrating immune cell presence and PD-L1 protein expression in both tumor and immune cells; 43 blocks were adenocarcinoma and 112 blocks were squamous cell carcinoma tumors. These tissue blocks were obtained from 12 different vendors. The number of blocks from the top five vendors are as follows: 58, 37, 36, 17, and 8. No one vendor provided more than 35% of the blocks. There was insufficient evaluable tumor material available to support PD-L1 biomarker correlative analysis in patients enrolled in the cervical cancer expansion cohorts of the phase I study. Among 20 patients in the cervical cancer expansion cohorts, no pre-treatment sample was submitted for six patients and the pre-treatment sample had insufficient tumor cells for one patient. Table 1 lists the results of PD-L1 IHC analysis and best overall response status for the remaining 13 patients.

TABLE 1
Summary of PD-L1 testing on available pre-treatment samples from cervical
cancer patients

| TC % PD-L1 | Immune cell | IC % PD-L1 | | Best Overall |
|---|---|---|---|---|
| positive | Presence | positive | Histology | Response |
| 70 | 5 | 2 | Adenocarcinoma | PD |
| <1 | 10 | 9 | Adenocarcinoma | PD |
| <1 | 5 | <1 | Adenocarcinoma | SD |
| <1 | 5 | <1 | Adenocarcinoma | SD |
| 8 | 1 | <1 | Adenosquamous cell carcinoma | SD |
| <1 | 10 | 7 | Squamous cell carcinoma | PD |
| <1 | 5 | 1 | Squamous cell carcinoma | PD |
| <1 | 10 | 8 | Squamous cell carcinoma | PD |
| <1 | 10 | 3 | Squamous cell carcinoma | SD |
| 15 | 5 | 4 | Squamous cell carcinoma | SD |
| 20 | 10 | 7 | Squamous cell carcinoma | SD |
| 30 | 10 | 7 | Squamous cell carcinoma | SD |
| <1 | 10 | 4 | Squamous cell carcinoma | SD |

IC, immune cell;
PD, progressive disease;
SD, stable disease;
TC, tumor cell.

PD-L1 immunohistochemistry staining was conducted with the Ventana PD-L1 (SP263) rabbit monoclonal primary antibody (Roche Diagnostics; AZ) according to the manufacturer instructions for use. PD-L1 scoring was reported as percentage of tumor cells with any membrane staining above background (TC %) or the percentage of tumor-associated immune cells with staining at any intensity above background (IC %). Any tumor-associated immune cell in a mixed inflammatory setting and not part of necrosis was included in calculating the percent of tumor area occupied by tumor-associated immune cells (Immune Cells Present, ICP). To facilitate analysis and interpretation, cut-off values to group PD-L1 expression and immune cell presence were determined by organizing the distribution into discrete units.

PD-L1 mRNA expression was plotted for squamous and non-squamous cervical cancer tumors in The Cancer Genome Atlas (TOGA). Transcripts per million (TPM) of cervical tumors were plotted using OmicSoft ArrayStudio software, version 10.0.1.50. PD-L1 mRNA expression results are in whole based upon data generated by the TOGA Research Network (https://www.cancer.gov/tcga).

Results

Patients—Ten patients were enrolled in each expansion cohort. The median age was 55.0 years (range, 31.0-76.0) and 51.5 years (range, 29.0-65.0) in the monotherapy and combination therapy cohorts, respectively. Baseline characteristics were similar across the two cohorts and are summarized in Table 2. Ten patients had squamous histology, eight patients had adenocarcinoma, one patient had adenosquamous histology, and one patient had mucinous carcinoma histology. At the time of data cut-off, among patients receiving cemiplimab monotherapy, one patient had completed planned treatment (48 weeks) and nine patients had discontinued treatment, mainly due to disease progression or recurrence (n=8). In patients receiving cemiplimab+hfRT, all 10 patients had discontinued treatment, mainly due to disease progression or recurrence (n=8).

TABLE 2
Patient demographics and baseline characteristics

| | Cemiplimab monotherapy | | Cemiplimab + hfRT | |
|---|---|---|---|---|
| | (n = 10) | | (n = 10) | |
| Median age, years (range) | 55.0 | (31.0-76.0) | 51.5 | (29.0-65.0) |
| Race, n (%) | | | | |
| White | 9 | (90.0) | 8 | (80.0) |
| Black or African American | 0 | | 1 | (10.0) |
| Asian | 0 | | 1 | (10.0) |
| Not reported | 1 | (10.0) | 0 | |
| ECOG performance status, n (%) | | | | |
| 0 | 4 | (40.0) | 2 | (20.0) |
| 1 | 6 | (60.0) | 8 | (80.0) |
| Tumor histology, n (%) | | | | |
| Squamous | 4 | (40.0) | 6 | (60.0) |
| Adenocarcinoma | 6 | (60.0) | 2 | (20.0) |
| Other | 0 | | 2^{a} | (20.0) |
| Prior cancer-related systemic therapy, | 10 | (100.0) | 10 | (100.0) |
| n (%)^{b} | | | | |
| Platinum compounds | 10 | (100.0) | 10 | (100.0) |
| Taxanes | 10 | (100.0) | 10 | (100.0) |
| Bevacizumab | 7 | (70.0) | 7 | (70.0) |
| Other^{c} | 10 | (100.0) | 3 | (30.0) |
| Prior cancer-related radiotherapy, n (%) | 10 | (100.0) | 8 | (80.0) |

^{a}Other histology was adenosquamous cell carcinoma in one patient and mucinous carcinoma in one patient.
^{b}Chemical/pharmacological/therapeutic subgroup code was not available for one patient receiving cemiplimab + hfRT
ECOG, Eastern Cooperative Oncology Group; hfRT, hypofractionated radiation therapy.

For patients receiving cemiplimab monotherapy, the median number of administered doses of cemiplimab was 4 (range, 2.0-23.0), with median duration of exposure of 8.1 weeks (range, 4.0-48.4) and median duration of follow-up of 5.6 months (range, 0.8-16.2). For patients receiving cemiplimab+hfRT, the median number of administered doses of cemiplimab was 8 (range, 1.0-17.0), median duration of exposure 16.0 weeks (range, 2.0-34.1) with a median follow-up of 3.76 months (range, 0.7-8.1).

Efficacy—One patient in each cohort (10%) experienced a partial response, with a duration of 11.2 months for the monotherapy cohort responder and 6.4 months for the combination therapy cohort responder. Both responders had squamous histology. Irradiated lesions were not included in the response assessments. Eight patients achieved best response of stable disease (SD): three patients (30%) in the monotherapy cohort and five patients (50%) in the combination therapy cohort (Table 3). Of these eight patients with best response of SD, four had squamous histology. Durable disease control, defined as the proportion of patients without progressive disease for 105 days, was 20.0% (95% CI: 2.5-55.6) in patients receiving cemiplimab monotherapy and 30.0% (95% CI: 6.7-65.2) in patients receiving cemiplimab+hfRT. Other clinical activity results regarding tumor response status are summarized in Table 3. Partial Response (PR) is determined if measurements of target lesions and new lesions are ≤30% of baseline. Progressive Disease (PD) is determined if measurements of target lesions and new lesions are ≤20% from the lowest measurements.

TABLE 3
Tumor response per investigator assessment

| | Cemiplimab monotherapy | | Cemiplimab + hfRT | |
|---|---|---|---|---|
| | (n = 10) | | (n = 10) | |
| ORR, % (95% CI) | 10.0 | (0.3-44.5) | 10.0 | (0.3-44.5) |
| Partial response, n (%) | 1 | (10.0) | 1 | (10.0) |
| Stable disease, n (%) | 3 | (30.0) | 5 | (50.0) |
| Progressive disease, n (%) | 5 | (50.0) | 4 | (40.0) |
| NE^{a}, n (%) | 1 | (10.0) | 0 | |
| Disease control rate, % (95% CI)^{b} | 40.0 | (12.2-73.8) | 60.0 | (26.2-87.8) |
| Durable disease control rate, | 20.0 | (2.5-55.6) | 30.0 | (6.7-65.2) |

| | | |
|---|---|---|
| % (95% CI)^{c} | | |
| Observed time to response, months^{d} | 1.8 | 1.8 |
| DOR, months^{b} | 11.2 | 6.4 |

^{a}NE response includes missing and unknown tumor response.
^{b}Defined as proportion of patients with objective respose or stable disease.
^{c}Defined as the proportion of patients with objective response or stable disease without progression for at least 16 weeks, measured at least 105 days to account for scheduling windows in the protocol.
^{d}Based on one patient in each group who experienced objective tumor response.
CI, confidence interval;
DOR, duration of response;
IQR, interquartile range;
NE, not evaluable;
ORR, objective response rate.

Kaplan-Meier estimation of median PFS was 1.9 (95% CI: 1.0-9.0) months in patients receiving cemiplimab monotherapy and 3.6 (95% CI: 0.6-5.7) months in patients receiving cemiplimab+hfRT. Kaplan-Meier estimation of median OS was 10.3 (95% CI: 2.1—not evaluable [NE]) months in patients receiving cemiplimab monotherapy and 8.0 (95% CI: 1.7—NE) months in patients receiving cemiplimab+hfRT.

Safety—TEAEs of any grade were reported in 9 and 10 patients in the monotherapy and combination therapy cohorts, respectively, regardless of treatment attribution. The most common TEAEs were diarrhea in 35% (7/20), fatigue in 25% (5/20), and hypokalemia in 25% (5/20) of patients enrolled in both cohorts combined. Of patients receiving cemiplimab monotherapy, four (40.0%) patients experienced grade TEAEs. Of the patients receiving cemiplimab+hfRT, four (40.0%) patients experienced grade TEAEs. No patients in either cohort discontinued treatment due to TEAEs.

One death, due to pneumonitis, was reported in a patient receiving cemiplimab+hfRT and was considered treatment-related. However, other co-morbidities with potential influence on the patient's outcome included possible diffuse malignant pulmonary involvement, pericardial tumor potentially impacting diastolic filling pressures, and decreased general condition, along with medical history elements such as tobacco use and chronic obstructive pulmonary disease. The lesion selected for palliative radiation therapy (RT) in this patient was on the pericardium. At time of death from pneumonitis, the patient's overall tumor response was partial response. Another patient also experienced grade 3 pneumonitis, and this resolved with treatment that included steroids and empiric antibiotics. One patient with baseline fatigue experienced grade 3 immune-related myalgia and grade 2 immune-related hypothyroidism that was associated with intermittent grade 2 and 3 fatigue. Myalgia resolved with steroid treatment. Treatment-related TEAEs (Grade 3) occurred in 10% (1/10) patients in the monotherapy cohort and 30% (3/10) patients in the combination therapy cohort, as summarized in Table 4.

TABLE 4
Summary of treatment-related adverse events

| | Cemiplimab monotherapy | | | | Cemiplimab + hfRT | | | |
|---|---|---|---|---|---|---|---|---|
| | (n = 10) | | | | (n = 10) | | | |
| Treatment-related TEAE, n (%) | Any grade | | Grade ≥ 3 | | Any grade | | Grade ≥ 3 | |
| Any | 7 | (70.0) | 1 | (10.0) | 6 | (60.0) | 3 | (30.0) |

| Most common^{a} | | | | | | | |
|---|---|---|---|---|---|---|---|
| Fatigue | 3 | (30.0) | 1 | (10.0) | 1 | (10.0) | 0 |
| Diarrhea | 2 | (20.0) | 0 | (0) | 3 | (30.0) | 0 |

| Hypothyroidism | 2 | (20.0) | 0 | 0 | | 0 | |
|---|---|---|---|---|---|---|---|
| Pneumonitis | 1 | (10.0) | 0 | 2 | (20.0) | 2 | (20.0) |
| Hyponatremia | 1 | (10.0) | 0 | 1 | (10.0) | 1 | (10.0) |

| | | | | | | |
|---|---|---|---|---|---|---|
| Myalgia | 1 | (10.0) | 1 | (10.0) | 0 | 0 |

^{a}Occurred in two or more patients in either cohort of any grade, or grade ≥ 3 in any patient; ordered by overall frequency in cemiplimab monotherapy cohort.

Protein and PD-L1 mRNA expression in cervical cancer—The observation that responses occurred in patients with squamous histology prompted exploration of potential associations between PD-L1 expression and histology in cervical cancer. There were insufficient samples available from study patients, and therefore, archived cervical cancer specimens from other sources were interrogated. As shown in Table 5, among 155 tumor samples analyzed by immunohistochemistry, tumor PD-L1 expression was undetectable (<1%) in 69.8% (30/43) of adenocarcinomas and 40% (45/112) of squamous cell carcinoma samples. PD-L1 expression in immune cells was undetectable (<1%) in 30.2% (13/43) of adenocarcinomas in contrast to 4.5% (5/112) in squamous cell carcinomas. Combined enrichment analysis demonstrated that both immune cell presence and expression of PD-L1 in tumor cells and immune cells were more common in squamous cell carcinomas than in adenocarcinoma tumors.

TABLE 5
Presence of immune cells and frequencies of PD-L1 expression in
tumor and immune cells in cervical SCC and adenocarcinoma

| | | | Immune | | Immune | |
|---|---|---|---|---|---|---|
| | Tumor | | cell | | cell | |
| | staining, | Distribution, | staining, | Distribution, | present, | Distribution, |
| | % | n (%) | % | n (%) | % | n (%) |
| Adenocarcinoma | <1 | 30 (69.8) | <1 | 13 (30.2) | <1 | 29 (67.4) |
| (n = 43) | <25 | 11 (25.6) | <20 | 18 (41.9) | <10 | 7 (16.3) |
| | <50 | 0 (0.0) | <40 | 10 (23.3) | <25 | 7 (16.3) |
| | ≥50 | 2 (4.7) | ≥41 | 2 (4.7) | ≥25 | 0 (0.0) |
| SCC (n = 112) | <1 | 45 (40.2) | <1 | 5 (4.5) | <1 | 29 (25.9) |
| | <25 | 36 (32.1) | <20 | 55 (49.1) | <10 | 45 (40.2) |
| | <50 | 16 (14.3) | <40 | 42 (37.5) | <25 | 34 (30.4) |
| | ≥50 | 15 (13.4) | ≥41 | 10 (8.9) | ≥25 | 4 (3.6) |

PD-L1, programmed death ligand-1; SCC, squamous cell carcinoma

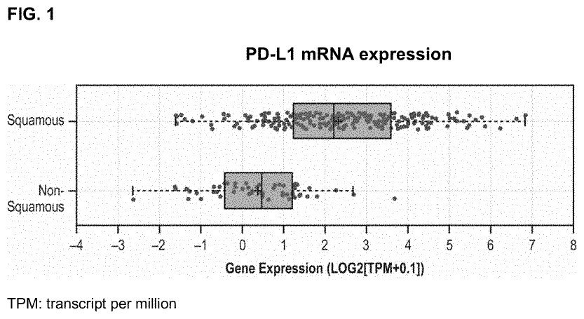

Potential associations between PD-L1 expression and tumor histology in cervical cancer were also explored at the mRNA level in TOGA samples. PD-L1 mRNA expression was greater in squamous versus non-squamous cervical cancer samples. The median and mean TPM of PD-L1 mRNA for squamous cervical tumors (n=253) were 4.5 and 5.0, respectively. The median and mean for non-squamous cervical tumors (n=53) were 1.3 and 1.2, respectively. (FIG. 1). Additional TOGA analyses for expression of selected genes (PD-1, PD-L1, CD8A) showed that cervical cancer clusters with other solid tumor types for which anti-PD-1 therapy improves overall survival (OS), such as melanoma, non-small cell lung cancer, renal clear cell carcinoma, and head and neck squamous cell carcinoma.

Discussion

Clinical activity results of these expansion cohorts demonstrate that treatment with cemiplimab induces responses and clinical benefit among recurrent or metastatic cervical cancer patients. This supports the clinical activity signal observed in the dose escalation cohort of the cemiplimab first-in-human study, in which two of three cervical cancer patients had durable responses. (Papadopoulos et al., Clin Cancer Res (December 2019) Epub). The sum total equates to a 17% objective response rate among cervical cancer patients enrolled in the phase I study (4/23 combined; 2/3 responding patients in dose escalation plus 2/20 in expansion cohorts). All responders had squamous histology. The safety results observed here are consistent with what has been observed in other studies of cemiplimab and other inhibitors of the PD-1/PD-L1 axis. There was no apparent improvement to objective response rate (ORR) from adding hfRT after the initiation of cemiplimab treatment. In fact, one of the cervical squamous patients who had objective response to cemiplimab+hfRT in dose escalation subsequently had disease recurrence after completion of planned treatment, and experienced complete response to retreatment with cemiplimab monotherapy.

In our analysis of cervical cancer specimens independent of the phase I trial, the combination of both high PD-L1 protein expression and immune cell presence was enriched in squamous relative to adenocarcinomas. These results confirm and extend a prior report which used a different anti-PD-L1 antibody and found higher expression in squamous than in adenocarcinoma cervical cancer samples (Heeren et al., Cancer Immunol Res 3 (2015) 48-58). The study of the present example supports these observations by reporting that in TOGA cervical cancer samples, PD-L1 mRNA expression is greater in squamous than in non-squamous samples. Increased PD-L1 expression among squamous tumors at both the protein and mRNA level indicates that mechanisms of immune-evasion may differ between squamous and non-squamous cervical cancers and may impact clinical response to immunotherapy. Expression levels of PD-L1 mRNA in squamous versus non-squamous histologies were not previously described.

The importance of the PD-1/PD-L1 axis in squamous cervical cancer is also supported by studies with other PD-1 inhibitors. In the multi-cohort study KEYNOTE-158, ORR in the cervical cancer cohort was 12.2% (12/98) with pembrolizumab, and 11 of the 12 observed responses were in patients with squamous histology. (Papadopoulos et al., Clin Cancer Res (December 2019) Epub). In CheckMate 358, a phase I/II study in for patients with squamous histology only, the objective response rate was 26.3% (5/19) in cervical cancer with nivolumab. (Naumann et al., J Clin Oncol 37 (2019) 2825-2834). Another study, with a patient population of 60% squamous histology, also reported a 4% response rate to nivolumab. (Santin et al., Gynecol Oncol 157 (2020) 161-166). Although these observations are directionally consistent with the hypothesis that the efficacy of PD-1 blockade in cervical cancer may partition with squamous histology, it is not possible to draw conclusions from small cross-study comparisons. The present data, and other studies reviewed here, do not exclude the possibility that some patients with non-squamous cervical cancers may also benefit from immunotherapy.

Although most patients with recurrent or metastatic cervical cancer do not experience objective responses with PD-1 blockade, the potential for durable responses (or durable stable disease) could lead to meaningful survival benefits. The current study is too small to provide robust estimates of OS.

Analysis of gene expression data for selected genes (PD-1, PD-L1, CD8A) in TOGA demonstrates that cervical cancer clusters with other solid tumor types for which anti-PD-1 therapy improves overall survival, such as melanoma, non-small cell lung cancer, renal clear cell carcinoma, and head and neck squamous cell carcinoma. (Trivedi et al., Clin Adv Hematol Oncol 13 (2015) 858-868; Ferris et al., N Engl J Med 375 (2016) 1856-1867; Lee et al., Cancer J 22 (2016) 92-95; Chamoto et al., Int J Clin Oncol (2020); Gellrich et al., J Clin Med 9 (2020) 223; Chae et al., J Immunother Cancer 6 (2018) 39). Additionally, TOGA analysis of 36 variables in 21 tumor types found that CD8+ T cell abundance, PD-1 gene expression, and tumor mutational burden were the three most predictive variables of objective response to anti-PD-1/PD-L1 therapy. (Lee et al., JAMA Oncol 5 (2019) 1614-1618). Two of these variables (CD8+ T cell abundance, PD-1 gene expression) are consistent with the TOGA analysis in the current report that clusters cervical cancer with other immunotherapy-responsive tumors. The third variable, tumor mutation burden, may also conbribute to the efficacy of PD-1 blockade on some cervical cancer patients. In exploratory analyses of expansion cohorts in a phase II study of pembrolizumab (KEYNOTE-158), cervical squamous cell cancer was among the tumor types in which high tissue tumor mutation burden (defined as >10 mutations/megabase) was associated with increased efficacy. (Marabelle et al., Annals of Oncology 30 (2019) v477-v478). Thus, the TOGA analyses presented here further support the identification of cervical cancer as a tumor type for which cemiplimab therapy should be beneficial.

A randomized phase III trial is ongoing in second-line or greater metastatic cervical cancer patients, comparing cemiplimab versus investigator's choice of chemotherapy (NCT03257267). The primary analysis for OS is hierarchical, first for patients with squamous histology and then for all patients (squamous, adenocarcinoma, or adenosquamous histology). Associations between PD-L1 expression, efficacy, and histology is explored.

### CONCLUSION

Among patients with recurrent or metastatic cervical cancer who were resistant to or intolerant of platinum and taxane doublet chemotherapy, cemiplimab demonstrated clinical benefit and a safety profile similar to those observed with other PD-1 inhibitors. The results from the cemiplimab trials in conjunction with data with other anti-PD-1 agents suggest that efficacy is associated with histology in cervical cancer. Furthermore, the potential association between histology and efficacy of PD-1 inhibition is supported indirectly by analyses of cervical cancer specimens from other sources in which PD-L1 protein and mRNA expression is greater in squamous than in non-squamous histologies. A phase III randomized trial of cemiplimab versus investigator's choice of chemotherapy is ongoing, and the primary overall survival hierarchical analysis is first in patients with squamous histology.

### Example 2: Clinical Trial of Cemiplimab Versus Chemotherapy in Recurrent or Metastatic Cervical Carcinoma

This study is an open-label, randomized, phase 3 trial of cemiplimab versus investigator's choice (IC) chemotherapy in patients with recurrent or metastatic cervical cancer that has progressed after platinum-containing chemotherapy.

Study Objectives

A primary objective of the study is to compare OS for patients with recurrent or metastatic cervical cancer who have histology of squamous cell carcinoma (SCC) and who have any eligible histology, treated with either cemiplimab or investigator's choice (IC) chemotherapy. Secondary objectives of the study performed among SCC patients and among all eligible histologies (SCC, adenocarcinoma or adenosquamous carcinoma) include: (1) to compare progression-free survival (PFS) of cemiplimab versus IC chemotherapy; (2) to compare objective response rate (ORR) (partial response [PR]+CR) of cemiplimab versus IC chemotherapy per Response Evaluation Criteria in Solid Tumors (RECIST) 1.1; (3) to compare the duration of response (DOR) of cemiplimab versus IC chemotherapy; (4) to compare the safety profiles of cemiplimab versus IC chemotherapy by describing adverse events (AE); and (5) to compare quality of life (QOL) for patients treated with cemiplimab versus IC chemotherapy using European Organization for Research and Treatment of Cancer Quality of Life Questionnaire-Core 30 (EORTC QLQ-C30).

Study Population

The study is enrolling women ≥18 years old with recurrent, persistent, and/or metastatic cervical cancer that has progressed after platinum-containing chemotherapy given to treat recurrent or metastatic cervical cancer. Patients who have only received prior platinum-based therapy concurrently with radiation therapy for localized disease are not eligible.

Rationale for patient population: The patients in this study have recurrent or metastatic cervical cancer that has progressed after platinum-containing chemotherapy. A prior study established the efficacy of first line-therapy for recurrent or metastatic cervical cancer with the regimen of platinum+paclitaxel+bevacizumab. (Tewari et al., N Engl J Med, 370 (2014) 734-743). There is no standard-of-care regimen in the second line setting. Agents that may be considered in this setting are the IC options in this study: pemetrexed, topotecan, irinotecan, gemcitabine, and vinorelbine. Despite the availability of various chemotherapy options, patients treated with these agents for cervical cancer have a median survival time of approximately 7 months.

A concept of “platinum-refractory” disease has been described in the cervical cancer literature, and is related to time since prior platinum therapy. (Nishino et al., Clin Cancer Res., August 2016; McLachlan et al., Clinical Oncology 2016; Tanioka et al., Cancer Chemother Pharmacol (2011) 68:337-342). However, it is not typical clinical practice to re-treat cervical cancer patients with platinum-based chemotherapy if they have already received it in the setting of recurrent or metastatic disease. The chemotherapy regimens in the control arm of this study represent the current treatment options for cervical cancer patients who have received prior platinum in the setting of recurrent or metastatic disease. Regardless of the time interval between prior platinum therapy (for recurrent or metastatic cervical cancer) and subsequent progression, such patients have unmet medical need and are appropriate for consideration of the clinical study. Platinum-therapy given in other settings (e.g., concurrent with radiation therapy as part of curative-intent therapy, after radiation [or chemoradiation] as adjuvant treatment in a patient with no evidence of disease) does not satisfy the eligibility requirement regarding prior platinum therapy in this study.

The term “persistent disease” is sometimes used to refer to disease for which there was never documentation of complete resolution after chemoradiation. For such patients, first line therapy is the same as that for patients with recurrent or metastatic disease (i.e., platinum+paclitaxel ±bevacizumab). As a convention in this study, patients with persistent disease are considered included in the category of “recurrent or metastatic” disease any time that term is used herein.

The study population will have received prior paclitaxel and prior bevacizumab, refused such treatment, or been unsuitable for such treatment (or, in the case of bevacizumab, not had access). For patients who received prior paclitaxel and/or prior bevacizumab, disease progression at any time after prior paclitaxel and/or bevacizumab is an acceptable reason for discontinuation of paclitaxel and/or bevacizumab.

The study design is a randomized comparison of cemiplimab versus IC chemotherapy, with an OS endpoint. For patient populations in which there is no widely accepted standard of care, and in which randomization to a placebo or best-supportive care arm is considered unethical or unfeasible, health authorities have accepted IC as a comparator in studies that have led to regulatory approvals based on OS endpoints (Donoghue et al., Clin Canc Res, 2012; 18:1496-1505; Ferris et al., New Engl J Med, October 2016 Epub). Blinding is not practical in the current study due to differences in schedule and differences in AE profiles between cemiplimab and the IC options (i.e., immune-related adverse events [irAEs] with cemiplimab and white blood cell count suppression with chemotherapy).

Overall survival directly measures clinical benefit, is not biased, and is not a surrogate endpoint. As such, OS has been selected as the primary endpoint, and this open-label study compares cemiplimab versus IC chemotherapy in patients with cervical cancer who have progressed after prior treatment with a platinum containing regimen that was given in the recurrent or metastatic disease setting.

Inclusion Criteria: A patient must meet the following criteria to be eligible for inclusion in the study: (1) recurrent, persistent, and/or metastatic cervical cancer with squamous cell histology, for which there is not a curative-intent option (surgery or radiation therapy with or without chemotherapy) (Note: Patients with adenocarcinoma and adenosquamous carcinoma histologies were also enrolled according to the original protocol); (2) tumor progression or recurrence after treatment with platinum therapy (must have been used to treat metastatic, persistent, or recurrent cervical cancer); (3) patient must have measurable disease as defined by RECIST 1.1. Measurable disease is defined as at least one lesion that can be accurately measured in at least 1 dimension (longest dimension to be recorded). Each lesion must be mm when measured by computed tomography (CT), magnetic resonance imaging (MRI), or caliper measurement by clinical exam or must be ≥20 mm when measured by chest x-ray. Lymph nodes must be >15 mm in short axis when measured by CT or MRI; (4) Eastern Cooperative Oncology Group (ECOG) performance status ≤1; (5) ≥18 years old; (6) Hepatic function: total bilirubin ≤1.5× upper limit of normal (ULN; if liver metastases ≤3×ULN); transaminases ≤3×ULN (or ≤5.0×ULN, if liver metastases); alkaline phosphatase ≤2.5×ULN (or ≤5.0×ULN, if liver or bone metastases); (7) Renal function: Serum creatinine ≤1.5×ULN or estimated creatinine clearance >45 mL/min; (8) Bone marrow function: Hemoglobin ≥9.0 g/dL; Absolute neutrophil count (ANC) ≥1.5×10⁹/L; Platelet count ≥75×10⁹/L; (9) Anticipated life expectancy >12 weeks; (10) Willing and able to comply with clinic visits and study-related procedures; (11) Provide signed informed consent; (12) Able to understand and complete study-related questionnaires; (13) Patients must meet at least one of the following criteria regarding prior bevacizumab therapy: (a) Received prior bevacizumab-containing therapy, which was discontinued due to progression of disease; (b) Received prior bevacizumab-containing therapy, which was discontinued due to toxicity; (c) Was deemed unsuitable for prior bevacizumab therapy for one of the following reasons: (i) unacceptable risk of fistula formation, (ii) poorly controlled hypertension, (iii) “low risk” disease according to the Moore Criteria (Tewari et al., Clin Cancer Res 2015; 21(24)); (d) Refused prior bevacizumab therapy; (e) Did not have access to bevacizumab therapy due to logistical reasons (e.g., lived in a region in which bevacizumab was not commercially available for patients with cervical cancer, or did not have insurance coverage for bevacizumab); (14) Patients must meet at least one of the following criteria regarding prior paclitaxel therapy: (a) Received prior paclitaxel-containing therapy, which was discontinued due to progression of disease; (b) Received prior paclitaxel-containing therapy, which was discontinued due to toxicity; (c) Was deemed unsuitable for prior paclitaxel therapy for one of the following reasons: (i) neuropathy (ii) allergy to paclitaxel or its components; (d) Refused prior paclitaxel therapy.

Exclusion Criteria: A patient who meets any of the following criteria is excluded from the study: (1) Ongoing or recent (within 5 years) evidence of significant autoimmune disease that required treatment with systemic immunosuppressive treatments, which may suggest higher risk for severe irAEs. The following are not exclusionary: vitiligo, childhood asthma that has resolved, type 1 diabetes, residual hypothyroidism that required only hormone replacement, or psoriasis that does not require systemic treatment; (2) Prior treatment with an agent that blocks the PD-1/PD-L1 pathway; (3) Prior treatment with other systemic immune-modulating agents that was (a) within fewer than 4 weeks (28 days) of the enrollment date, or (b) associated with irAEs of any grade within 90 days prior to enrollment, or (c) associated with toxicity that resulted in discontinuation of the immune-modulating agent. Examples of immune-modulating include therapeutic vaccines, cytokine treatments (other than granulocyte colony stimulating factor or erythropoietin), or agents that target cytotoxic T-lymphocyte antigen 4 (CTLA-4), 4-1BB (CD137), PI 3-K-delta, LAG3, or OX-40; (4) Known history of brain metastasis(es) that may be considered active (screening imaging of brain is not required unless there is clinical suspicion of brain metastases). Patients with previously treated brain metastases may participate provided that the lesions are stable (without evidence of progression for at least 6 weeks on imaging obtained during the screening period), there is no evidence of new or enlarging brain metastases, and the patient does not require any immunosuppressive doses of systemic corticosteroids for management of brain metastases within 4 weeks of the first dose of study drug (cemiplimab or IC chemo); (5) Immunosuppressive corticosteroid doses (>10 mg prednisone daily or equivalent) within 4 weeks prior to the first dose of study drug (cemiplimab or IC chemo); (6) Active bacterial, viral, fungal or mycobacterial infection requiring therapy, including known infection with human immunodeficiency virus (HIV), or active infection with hepatitis B virus (HBV) or hepatitis C virus (HCV); (7) History of pneumonitis within the last 5 years; (8) Any anticancer treatment (chemotherapy, targeted systemic therapy, photodynamic therapy), investigational, or standard of care, within 30 days of the initial administration of study drug (cemiplimab or IC chemo) or planned to occur during the study period (patients receiving bisphosphonates or denosumab are not excluded); (9) History of documented allergic reactions or acute hypersensitivity reaction attributed to antibody treatments; (10) Concurrent malignancy other than cervical cancer and/or history of malignancy other than cervical cancer within 3 years of date of first planned dose of study drug (cemiplimab or IC chemo), except for tumors with negligible risk of metastasis or death, such as adequately treated cutaneous squamous cell carcinoma or basal cell carcinoma of the skin or ductal carcinoma in situ of the breast. Patients with hematologic malignancies (eg, chronic lymphocytic leukemia) are excluded; (11) Any acute or chronic psychiatric problems that, in the opinion of the investigator, make the patient ineligible for participation; (12) Patients with a history of solid organ transplant (patients with prior corneal transplant(s) may be allowed to enroll after discussion with and approval from the medical monitor); (13) Any medical co-morbidity, physical examination finding, or metabolic dysfunction, or clinical laboratory abnormality that, in the opinion of the investigator, renders the patient unsuitable for participation in a clinical trial due to high safety risks and/or potential to affect interpretation of results of the study; (14) Pregnant or breastfeeding women; (15) Women of childbearing potential who are unwilling to practice highly effective contraception prior to the initial study drug treatment, during the study, and for at least 6 months after the last dose. Highly effective contraceptive measures include stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening; intrauterine device; intrauterine hormone-releasing system; bilateral tubal ligation; vasectomized partner; and or sexual abstinence; (16) Patients committed to an institution by virtue of an order issued by either the judicial or the administrative authorities is excluded from this study; (17) Prior treatment with idelalisib; (18) Prior treatment with live vaccines within 30 days of initial administration of study drug (cemiplimab or IC chemo). Patients must not be treated with live vaccines during the study and up to 5 half-lives following the last dose of study drug; (19) Patients with prior treatment on any clinical trial within 30 days of the initial administration of study drug. Non-interventional and observational trials are acceptable.

Study Variables

The primary endpoint of this study is OS, defined as the time from randomization to the date of death. A patient who has not died is censored at the last known date of contact.

Secondary endpoints include Progression-free survival (PFS) and Overall Response Rate (ORR). PFS is defined as the time from randomization to the date of the first documented tumor progression using RECIST 1.1, or death due to any cause. ORR is defined as the number of patients with a best overall response of confirmed Complete Response (CR) or Partial Response (PR) divided by the number of patients in the efficacy analysis set. Best overall response is defined as the best overall response between the date of randomization and the date of the first objectively documented progression or the date of subsequent anti-cancer therapy, whichever comes first.

The following definitions apply to target lesions: Complete Response (CR) is defined as the disappearance of all target lesions; any pathological lymph nodes (whether target or non-target) must have reduction in short axis to <10 mm (<1 cm). Partial Response (PR) is defined as at least a 30% decrease in the sum of the diameters of target lesions, taking as reference the baseline sum diameters. Progressive Disease (PD) is defined as at least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm (0.5 cm). Stable Disease (SD) is defined as neither sufficient shrinkage to qualify for PR nor sufficient increase to qualify for PD, taking as reference the smallest sum diameters while on study.

Other secondary endpoints of the study include Duration of Response (DOR) and Quality of Life (QOL). DOR is defined as the time between the date of first response (CR or PR) to the date of the first documented tumor progression (per RECIST 1.1) or death due to any cause. QOL is measured by the EORTC QLQ-C30. Further secondary endpoints include the safety and tolerability of cemiplimab. To evaluate the safety and tolerability of cemiplimab IC chemotherapy, the incidence of AEs, serious adverse events (SAEs), deaths, and laboratory abnormalities are assessed.

Study Design

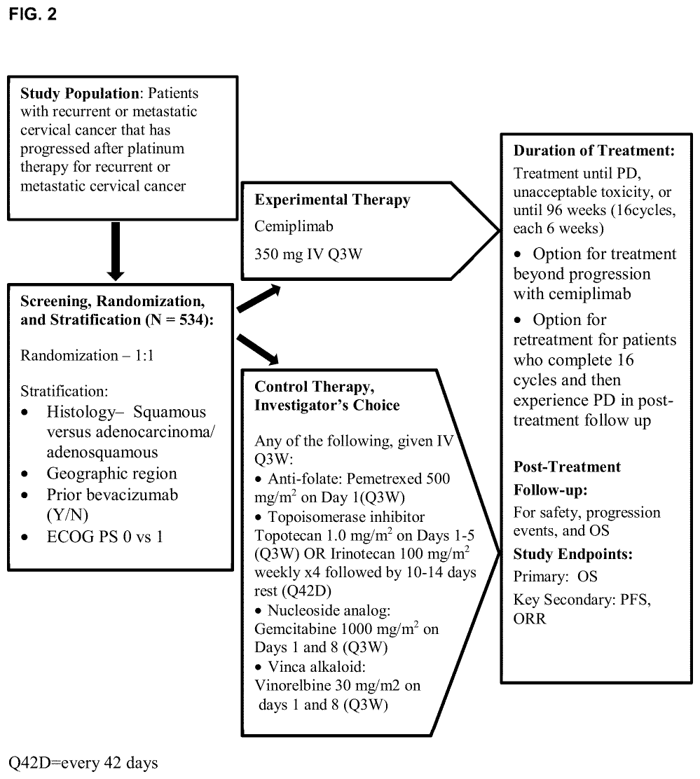

In this study, approximately 534 patients (436 of which have squamous cell carcinoma (SCC) histology) are randomized to either the experimental cemiplimab treatment arm or the IC of chemotherapy control treatment arm. The 436 SCC patients are randomized 1:1 (218 per treatment arm). In the experimental group, cemiplimab is administered as a flat dose of 350 mg Q3W. In the control group, IC chemotherapy options are in 4 classes: (1) antifolate pemetrexed, (2) topoisomerase 1 inhibitor—topotecan or irinotecan, (3) nucleoside analogue—gemcitabine, and (4) vinca alkaloid—vinorelbine. The only chemotherapy treatments allowed in the control arm are any of the 5 drugs that are listed as IC options. Other agents in these classes are not permitted in this study. FIG. 2 provides a schematic diagram of this study design.

The study includes 3 periods: screening, treatment, and follow-up. The screening period begins with the signing of the informed consent form (ICF). The screening period ends when the patient has been confirmed as fully eligible for the study and is randomized, or with confirmation that the patient is ineligible and is a screen failure. The treatment period begins within 5 days of randomization to 1 of the treatment arms. Cycle length is 6 weeks, and tumor imaging is planned to be conducted on day 42 (±7 days) of cycles 1-4, 6, 8, 10, 12, 14, and 16. Planned treatment is for up to 96 weeks. The treatment phase ends when the patient discontinues study therapy. There is no cross-over in this study. After completion of the treatment period, patients enter the follow-up period. After the follow-up period, patients are followed for survival. Study closeout procedures are implemented after the 331st OS event has been reported in squamous cell patients.

Treatments are provided to the patients in the study according to the schedule shown in Table 6.

TABLE 6
Treatments

| Treatment | Dose/Route/Schedule |
|---|---|
| Cemiplimab | administered IV as a flat dose of 350 mg Q3W, for up to 96 weeks of |
| | treatment |
| IC chemotherapy: | administered IV at a dose of 500 mg/m² Q3W, for up to 96 weeks of |
| Pemetrexed | treatment |
| IC chemotherapy: | administered IV at a dose of 1 mg/m² daily for 5 days, every 21 days, |
| Topotecan | for up to 96 weeks of treatment |
| IC chemotherapy: | administered IV at a dose of 100 mg/m² on days 1, 8, 15, and 22, |
| Irinotecan | followed by 10 to 14 days of rest, for a 42-day (6-week cycle), for up |
| | to 96 weeks of treatment. For patients enrolling in Japan, there are at |
| | least 14 days of rest before subsequent irinotecan administration |
| IC chemotherapy: | administered IV at a dose of 30 mg/m² on days 1 and 8, every 21 |
| Vinorelbine | days, for up to 96 weeks of treatment. |
| IC chemotherapy: | administered IV at a dose of 1000 mg/m² on days 1 and 8, every 21 |
| Gemcitabine | days, for up to 96 weeks of treatment |

The term “investigational product” (study drug) includes the experimental treatment cemiplimab and the IC chemotherapy treatments. In this study, the investigational products are: Cemiplimab (experimental group); Antifolate: Pemetrexed (an IC option in the control group); Topoisomerase inhibitor: Topotecan or irinotecan (IC options in the control group); Nucleoside analogue: Gemcitabine (an IC option in the control group); and Vinca alkaloid: Vinorelbine (an IC option in the control group). The only chemotherapy treatments allowed in the control arm are any of the 5 drugs that are listed above as IC options. Other agents in these classes are not permitted in this study.

Experimental Group Treatment (Cemiplimab): Open-label cemiplimab is supplied as a liquid in sterile, single-use vials. Each vial contains cemiplimab at a concentration of 50 mg/mL. Cemiplimab is administered in an outpatient setting as a 30-minute IV infusion. Each patient's dose is administered as a flat dose of 350 mg Q3W. The prepared infusion bag should be kept no more than 6 hours at room temperature, or no more than 24 hours at 2° C. to 8° C.

Control Group Treatments (Investigator's Choice): Patients assigned to the control arm receive one of the Investigator's Choice chemotherapy options, as follows: Antifolate: Pemetrexed, 500 mg/m² IV every 21 days, for up to 96 weeks of treatment. Vitamin B12 and folate support is provided according to standard of care with pemetrexed; Topoisomerase 1 inhibitor: Topotecan, 1 mg/m² daily IV for 5 days, every 21 days, for up to 96 weeks of treatment; or irinotecan 100 mg/m² IV weekly×4, followed by 10 to 14 days rest, for up to 96 weeks of treatment; Nucleoside analogue: Gemcitabine, 1000 mg/m² IV on days 1 and 8 and every 21 days, for up to 96 weeks of treatment; Vinca alkaloid: Vinorelbine 30 mg/m² IV on days 1 and 8 and every 21 days, for up to 96 weeks of treatment. For the IC options, doses are weight-based. For cycle 1/day 1, the investigator should use screening height and weight to calculate dose, but cycle 1/day 1 weight is also allowed to be used, per investigator discretion. Weight is measured at start of each cycle. If there is a ≥10% change in weight, the IC chemotherapy dose should be recalculated.

Procedures and Assessments

Tumor imaging (computed tomography [CT] or magnetic resonance imaging [MRI]) is performed to measure tumor burden and to characterize the efficacy profile of study treatments using response criteria. Every effort is made to collect survival data on all patients, including patients who withdraw from the study for any reason but have not withdrawn consent to collect survival information.

Physical examination, laboratory tests, vital signs, electrocardiogram (ECG), pregnancy test for women of childbearing potential, and recording of AEs and concomitant medications is performed to ensure patient safety and to characterize the safety profiles of study treatments.

Other assessments include: blood samples for pharmacokinetics (PK); blood samples to assess anti-cemiplimab antibodies; biomarkers (serum, plasma, tumor tissue); peripheral blood mononuclear cells; and quality of life assessments.

Procedures Performed at the Screening Visit: The following procedures are performed for the sole purpose of determining study eligibility or characterizing the baseline population: Serum β-HCG (result must be ≤72 hours before first dose); HBV, HCV, and HIV screening: hepatitis B surface antigen, hepatitis C positive RNA (positive hepatitis C antibody test will require hepatitis C RNA test to rule out active infection), HIV-1, or HIV-2 serum antibody; Documentation of pathologic confirmation of cervical cancer (squamous cell histology only); and Pathology material (formalin-fixed, paraffin-embedded [FFPE] block or 20 slides from the sample in the submitted pathology report).

Efficacy Procedures: For all patients, disease is measured radiologically according to RECIST 1.1 criteria. CT or MRI for tumor assessment is performed in screening, during treatment, and during follow-up. During the treatment period, tumor response assessments are performed at end of cycles 1 through 4, 6, 8, 10, 12, 14, and 16. During follow-up, tumor response assessments are performed at follow-up visits 1 and 2. The choice of whether the imaging is by CT or MRI is an investigator decision. Once the choice of CT scan or MRI has been made, subsequent assessments should be made using the same modality whenever possible. Whole-body (chest/abdomen/pelvis) imaging is performed at the baseline assessment and is strongly recommended at each response assessment. A CT or MRI of the neck should be performed in patients with metastases to neck. At a minimum, all radiologically measurable target lesions (RECIST 1.1) should be imaged at each response assessment. The same radiologic imaging modality should be used at each response assessment. Brain imaging—MRI brain with gadolinium (or CT brain with contrast, if MRI is not feasible) is performed in the screening period for patients with history of brain metastases, or for whom there is clinical suspicion of brain metastases. Patients with brain metastases that are “not active” who are enrolled in the study should have brain imaging at each response assessment, or sooner if there is clinical suspicion of worsening brain metastases during treatment.

Quality of Life Questionnaires: Patient-reported outcomes are measured using the validated patient self-administered EORTC QLQ-C30 questionnaire. Patients are asked to complete these questionnaires prior to any study procedures being performed at a given study visit (during the on-study/treatment and follow-up periods).

Concomitant Medications and Procedures

Any treatment administered, other than anti-cancer therapy, from the time of informed consent until 90 days after the last study treatment is considered concomitant treatment. This includes medications and other therapies for which administration started before the study and continue during the study, as well as any therapies started in the follow-up period to treat a study drug-related AE.

Prohibited Medications and Procedures: While participating in this study, a patient may not receive any anti-cancer treatment other than the treatment assigned at randomization: cemiplimab or IC of chemotherapy. Patients must not receive live vaccines during the study and for up to 5 half-lives after the last dose of study drug. Any other medication which is considered necessary for the patient's welfare, and which is not expected to interfere with the evaluation of the assigned treatment (cemiplimab or IC of chemotherapy), may be given at the discretion of the investigator. Patients using immunosuppressive doses (>10 mg per day of prednisone or equivalent) of systemic corticosteroids other than for corticosteroid replacement are not eligible for the study. For patients on the cemiplimab arm, it is recommended that patients do not receive systemic corticosteroids such as hydrocortisone, prednisone, prednisolone (Solu-Medrol®) or dexamethasone (Decadron®) at any time throughout the study except in the case of a life-threatening emergency and/or to treat an irAE.

Permitted Medications and Procedures: Physiologic replacement doses of systemic corticosteroids are permitted, even if >10 mg/day prednisone equivalents. A brief course of corticosteroids for prophylaxis (e.g., contrast dye allergy) or for treatment of non-autoimmune conditions (e.g., delayed-type hypersensitivity reaction caused by contact allergen) is permitted.

Radiation therapy is not part of the study regimen in either the experimental group or the control group. Patients for whom radiation therapy is planned are not eligible. If, during the course of the study, a patient develops a symptomatic lesion for which palliative radiation therapy is deemed appropriate by the investigator, this is deemed PD, and generally, the patient would be removed form study. Palliative radiation therapy may be allowed in certain circumstances in patients who have been on study for at least 24 weeks. Palliative radiation is only allowed to a non-target lesion in this study.

Results

It is expected that administration of cemiplimab leads to enhanced tumor regression in patients with cervical cancer with SCC histology. Such patients are expected to exhibit greater partial response and complete response, as well as significantly increased overall survival and overall response rate. It is also expected that such benefits will be achieved to an even greater degree in cervical cancer patients with SCC histology as compared to those observed in cervical cancer patients with adenocarcinoma histology.

### Example 3: Results of Phase 3 Study of Cemiplimab Versus Chemotherapy in Second Line (2L) Cervical Cancer

This example provides results obtained from the clinical trial described in Example 2. The study described in this example includes a total of 608 metastatic cervical cancer patients resistant to platinum-based chemotherapy (477 with squamous cell carcinoma (SCC); 131 with adenocarcinoma (AC)), with an Eastern Cooperative Oncology Group (ECOG) performance status of 0 or 1. The primary endpoint of this study was overall survival (OS). Secondary endpoints included progression free survival (PFS), overall response rate (ORR)/duration of response (DOR), safety, and quality of life.

Patients were treated with either: (i) cemiplimab, 350 mg IV Q3W or (ii) investigator's choice (IC) chemotherapy selected from pemetrexed, gemcitabine, topotecan (or irinotecan), or vinorelbine. This was a 90% power study. The median OS was 12 months for patients treated with cemiplimab, and 8.5 months for patients treated with chemotherapy. The hazard ratio (HR) per statistical design was 0.70. The minimum HR for statistical significance at final analysis was 0.80. The minimum HR for statistical significance in the second interim (85% events) was 0.77.

The demographics were balanced for total population, as summarized in Table 7, which provides demographics and baseline characteristics (full analysis set) for patients with SCC and non-SCC histology. The median age of 51.0 in the total patient population of this study is representative of the real world age of cervical cancer patients. Patients were allowed to enroll regardless of PD-L1 expression status.

TABLE 7
Patient Demographics

| | Cemiplimab | | Chemotherapy | | Total | |
|---|---|---|---|---|---|---|
| | (N = 304) | | (N = 304) | | (N = 608) | |
| Age (years) | | | | | | |
| N | 304 | | 304 | | 608 | |
| Mean (SC) | 51.1 | (11.59) | 51.2 | (11.77) | 51.1 | (11.67) |
| Median | 51.0 | | 50.0 | | 51.0 | |
| Q1:Q3 | 42.0:60.0 | | 43.0:59.0 | | 43.0:59.0 | |
| Min:Max | 22:81 | | 24:87 | | 22:87 | |
| Age Groups (years), n (%) | | | | | | |
| <65 | 269 | (88.5%) | 264 | (86.8%) | 533 | (87.7%) |
| ≥65 | 35 | (11.5%) | 40 | (13.2%) | 75 | (12.3%) |
| Age Groups (years), n (%) | | | | | | |
| <65 | 269 | (88.5%) | 264 | (86.8%) | 533 | (87.7%) |
| ≥65 and <75 | 30 | (9.9%) | 29 | (9.5%) | 59 | (9.7%) |
| ≥75 | 5 | (1.6%) | 11 | (3.6%) | 16 | (2.6%) |
| Geographic region, n (%) | | | | | | |
| North America | 32 | (10.5%) | 34 | (11.2%) | 66 | (10.9%) |
| Asia | 83 | (27.3%) | 83 | (27.3%) | 166 | (27.3%) |
| Rest of World | 189 | (62.2%) | 187 | (61.5%) | 376 | (61.8%) |
| ECOG performance status, n (%) | | | | | | |
| 0 | 142 | (46.7%) | 141 | (46.4%) | 283 | (46.5%) |
| 1 | 162 | (53.3%) | 163 | (53.6%) | 325 | (53.5%) |

The duration of treatment exposure for all patients was about 20 weeks. The results show a substantial advantage provided by cemiplimab as compared to chemotherapy (Tables 8-10), particularly with respect to overall survival (Table 8) of cervical cancer patients, including those with SCC and non-SCC histology.

TABLE 8
Overall Survival (OS) Results

| | SCC per IRT (N = 477) | | Total Population (N = 608) | | Adeno per IRT (N = 131) | |
|---|---|---|---|---|---|---|
| Number | Cemiplimab | Chemo | Cemiplimab | Chemo | Cemiplimab | Chemo |
| of patients | N = 239 | N = 238 | N = 304 | N = 304 | N = 65 | N = 66 |

Overall Survival (OS)

| Number of deaths (%) | 143 (59.8%) | 161 (67.6%) | 184 (60.5%) | 211 (69.4%) | 41 (63.1%) | 50 (75.8%) |
|---|---|---|---|---|---|---|
| Number of censored patients (%) | 96 (40.2%) | 77 (32.4%) | 120 (39.5%) | 93 (30.6%) | 24 (36.9%) | 16 (24.2%) |
| Median in month (95% Cl) | 11.1 (9.2, 13.4) | 8.8 (7.6, 9.8) | 12.0 (10.3, 13.5) | 8.5 (7.5, 9.6) | 13.3 (9.6, 17.6) | 7.0 (5.1, 9.7) |
| Reduced risk of death | 27% | | 31% | | 44% | |
| (vs. chemotherapy) | | | | | | |

| | | | |
|---|---|---|---|
| Hazard Ratio (95% Cl) | 0.727 (0.579, 0.914) | 0.685 (0.560, 0.838) | 0.556 (0.363, 0.853) |
| 1-Sided P-value | 0.00306 | 0.00011 | — |

TABLE 9
Progression Free Survival (PFS) Results

| | SCC per IRT (N = 477) | | Total Population (N = 608) | | Adeno per IRT (N = 131) | |
|---|---|---|---|---|---|---|
| Number | Cemiplimab | Chemo | Cemiplimab | Chemo | Cemiplimab | Chemo |
| of patients | N = 239 | N = 238 | N = 304 | N = 304 | N = 65 | N = 66 |

Progression Free Survival (PFS)

| Number of events (%) | 186 (77.8%) | 203 (85.3%) | 241 (79.3%) | 253 (83.2%) | 55 (84.6%) | 50 (75.8%) |
|---|---|---|---|---|---|---|
| Progressive disease (%) | 161 (67.4%) | 169 (71.0%) | 210 (69.1%) | 210 (69.1%) | 49 (75.4%) | 41 (62.1%) |
| Number of deaths (%) | 25 (10.5%) | 34 (14.3%) | 31 (10.2%) | 43 (14.1%) | 6 (9.2%) | 9 (13.6%) |
| Number of censored patients (%) | 53 (22.2%) | 35 (14.7%) | 63 (20.7%) | 51 (16.8%) | 10 (15.4%) | 16 (24.2%) |
| Median in month (95% Cl) | 2.8 (2.6, 4.0) | 2.9 (2.6, 3.9) | 2.8 (2.6, 3.9) | 2.8 (2.6, 3.3) | 2.7 (2.3, 4.0) | 2.8 (2.0, 3.2) |
| Reduced risk of progression | 31% | | 28% | | 15% | |
| (vs. chemotherapy) | | | | | | |

| | | | |
|---|---|---|---|
| Hazard Ratio (95% Cl) | 0.686 (0.559, 0.843) | 0.719 (0.599, 0.862) | 0.849 (0.574, 1.255) |
| 1-Sided P-value | 0.00014 | 0.00015 | — |

TABLE 10
Overall Response Rate (ORR) Results

| | SCC per IRT (N = 477) | | Total Population (N = 608) | | Adeno per IRT (N = 131) | |
|---|---|---|---|---|---|---|
| Number | Cemiplimab | Chemo | Cemiplimab | Chemo | Cemiplimab | Chemo |
| of patients | N = 239 | N = 238 | N = 304 | N = 304 | N = 65 | N = 66 |

Objective Response Rate (ORR)

| | | | | | | |
|---|---|---|---|---|---|---|
| ORR: CR + PR (%) | 42 (17.6%) | 16 (6.7%) | 50 (16.4%) | 19 (6.3%) | 8 (12.3%) | 3 (4.5%) |
| 95% Cl of ORR | (13%, 23%) | (3.9%, 10.7%) | (12.5%, 21.1%) | (3.8%, 9.6%) | (5.5%, 22.8%) | (0.9%, 12.7%) |

| | | | |
|---|---|---|---|
| Odds Ratio (95% Cl) | 3.002 (1.629, 5.53)* | 2.984 (1.707, 5.215)* | 2.894 (0.732, 11.445)* |
| 1-sided P-value | 0.00014* | 0.00004* | — |

Treatment-Emergent Adverse Events (TEAEs) were consistent with the known safety profile of cemiplimab, and superior to the chemotherapy safety profile, as summarized in Tables 11 and 12 (safety analysis set for patients with SCC and non-SCC histology). No new safety signals were observed.

TABLE 11
Summary of TEAEs

| | Cemiplimab | | Chemotherapy | |
|---|---|---|---|---|
| | (N = 300) | | (N = 290) | |
| Number of TEAEs | 1969 | | 2356 | |
| Number of NCI grade 3/4/5 TEAEs | 299 | | 385 | |
| Number of serious TEAEs | 147 | | 133 | |
| Number of patients with any TEAE, n (%) | 265 | (88.3%) | 265 | (91.4%) |
| Number of patients with any NCI grade 3/4/5 TEAE, n (%) | 135 | (45.0%) | 155 | (53.4%) |
| Number of patients with any serious TEAE, n (%) | 89 | (29.7%) | 78 | (26.9%) |
| Number of patients who discontinued study treatment due | 26 | (8.7%) | 15 | (5.2%) |
| to TEAEs, n (%) | | | | |
| Number of patients with any TEAE leading to a dose | 75 | (25.0%) | 114 | (39.3%) |
| interruption/delay, n (%) | | | | |
| Number of patients with any TEAE leading to a dose | 0 | | 58 | (20.0%) |
| reduction, n (%) | | | | |
| Number of patients with any TEAE resulting in death, n (%) | 5 | (1.7%) | 2 | (0.7%) |

TABLE 12
TEAEs-Total Population (all grades in cemiplimab ≥10%), No New Safety Signals
All Treatment-Emergent Adverse Events by Preferred Term and NCI Grade
(Safety Analysis Set)
Patients with SCC and Non-SCC Histology

| | Cemiplimab | | | | Chemotherapy | | | |
|---|---|---|---|---|---|---|---|---|
| | (N = 300) | | | | (N = 290) | | | |
| Preferred Term, n (%) | All Grades | | Grades 3/4/5 | | All Grades | | Grades 3/4/5 | |
| Number of patients with any TEAE, n (%) | 265 | (88.3%) | 135 | (45.0%) | 265 | (91.4%) | 155 | (53.4%) |
| Anemia | 75 | (25.0%) | 36 | (12.0%) | 129 | (44.5%) | 78 | (26.9%) |
| Nausea | 55 | (18.3%) | 1 | (0.3%) | 97 | (33.4%) | 6 | (2.1%) |
| Fatigue | 50 | (16.7%) | 4 | (1.3%) | 45 | (15.5%) | 4 | (1.4%) |
| Vomiting | 48 | (16.0%) | 2 | (0.7%) | 68 | (23.4%) | 7 | (2.4%) |
| Constipation | 45 | (15.0%) | 0 | | 59 | (20.3%) | 1 | (0.3%) |
| Decreased appetite | 45 | (15.0%) | 1 | (0.3%) | 46 | (15.9%) | 2 | (0.7%) |
| Pyrexia | 35 | (11.7%) | 1 | (0.3%) | 61 | (21.0%) | 0 | |
| Urinary tract infection | 35 | (11.7%) | 15 | (5.0%) | 25 | (8.6%) | 8 | (2.8%) |
| Asthenia | 33 | (11.0%) | 7 | (2.3%) | 44 | (15.2%) | 3 | (1.0%) |
| Back pain | 33 | (11.0%) | 4 | (1.3%) | 25 | (8.6%) | 2 | (0.7%) |
| Diarrhoea | 32 | (10.7%) | 3 | (1.0%) | 39 | (13.4%) | 4 | (1.4%) |

| | | | | | | | |
|---|---|---|---|---|---|---|---|
| Arthralgia | 31 | (10.3%) | 1 | (0.3%) | 8 | (2.8%) | 0 |

The foregoing results show that cemiplimab substantially reduced the risk of death by in patients with second-line recurrent or metastatic cervical cancer, compared to chemotherapy. This is the largest randomized Phase 3 clinical trial conducted in advanced cervical cancer. Cemiplimab is the first medicine to demonstrate an overall survival benefit in women with recurrent or metastatic cervical cancer following progression on platinum-based chemotherapy in a Phase 3 trial.

Compared to chemotherapy, cemiplimab decreased the risk of death by 31% (HR=0.69; CI: 0.56-0.84, p=0.003). Median survival was 12 months for patients receiving cemiplimab (n=304) compared to 8.5 months for patients receiving chemotherapy (n=304). No new cemiplimab safety signals were observed.

Recurrent or metastatic cervical cancer is notoriously difficult-to-treat and has no standard of care after first-line chemotherapy. This trial showed that cemiplimab helped patients with recurrent or metastatic cervical cancer live longer after chemotherapy failed, regardless of their PD-L1 status.

### Example 4: Results of Phase 3 Study of Cemiplimab Versus Chemotherapy in Recurrent or Metastatic Cervical Cancer

This example provides updated results over those shown in Example 3, and were obtained from the clinical trial described in Example 2. The study described in this example includes a total of 608 women with recurrent or metastatic cervical cancer who progressed after first-line platinum-based chemotherapy. Patients were enrolled regardless of PD-L1 expression status and received cemiplimab 350 mg intravenously every 3 weeks or investigator's choice (IC) chemotherapy, up to 96 weeks. IC chemotherapy options were pemetrexed, vinorelbine, gemcitabine, irinotecan, or topotecan. Patients were stratified by histology (squamous cell carcinoma (SCC)/adenocarcinoma or adenosquamous (AC)). Patient demographics are shown in Table 13.

Table 13
Summary of Patient Demographics and Baseline Characteristics in
Overall Population (full analysis set)

| | Cemiplimab | | Chemotherapy | | Total | |
|---|---|---|---|---|---|---|
| | (N = 304) | | (N = 304) | | (N = 608) | |
| Age (years) | | | | | | |
| n | 304 | | 304 | | 608 | |
| Mean (SC) | 51.1 | (11.59) | 51.2 | (11.77) | 51.1 | (11.67) |
| Median | 51.0 | | 50.0 | | 51.0 | |
| Q1:Q3 | 42.0:60.0 | | 43.0:59.0 | | 43.0:59.0 | |
| Min:Max | 22:81 | | 24:87 | | 22:87 | |
| Age Groups (years), | | | | | | |
| n (%) | | | | | | |
| <65 | 269 | (88.5) | 264 | (86.8) | 533 | (87.7) |
| ≥65 and <75 | 30 | (9.9) | 29 | (9.5) | 59 | (9.7) |
| ≥75 | 5 | (1.6) | 11 | (3.6) | 16 | (2.6) |
| Race, n (%) | | | | | | |
| White | 193 | (63.5) | 192 | (63.2) | 385 | (63.3) |
| Black or African | 9 | (3.0) | 12 | (3.9) | 21 | (3.5) |
| American | | | | | | |
| Asian | 88 | (28.9) | 88 | (28.9) | 176 | (28.9) |
| American Indian or | 2 | (0.7) | 1 | (0.3) | 3 | (0.5) |
| Alaska Native | | | | | | |
| Other | 8 | (2.6) | 4 | (1.3) | 12 | (2.0) |
| Unknown | 1 | (0.3) | 1 | (0.3) | 2 | (0.3) |
| Not Reported | 3 | (1.0) | 6 | (2.0) | 9 | (1.5) |
| Geographic region, | | | | | | |
| n (%) | | | | | | |
| North America | 32 | (10.5) | 34 | (11.2) | 66 | (10.9) |
| Asia | 83 | (27.3) | 83 | (27.3) | 166 | (27.3) |
| Rest of World | 189 | (62.2) | 187 | (61.5) | 376 | (61.8) |
| ECOG performance | | | | | | |
| status, n (%) | | | | | | |
| 0 | 142 | (46.7) | 141 | (46.4) | 283 | (46.5) |
| 1 | 162 | (53.3) | 163 | (53.6) | 325 | (53.5) |
| Histology / Cytology, | | | | | | |
| n (%) | | | | | | |
| Adenocarcinoma | 54 | (17.8) | 62 | (20.4) | 116 | (19.1) |
| Adenosquamous Cell | 10 | (3.3) | 9 | (3.0) | 19 | (3.1) |
| Carcinoma | | | | | | |
| Squamous Cell | 240 | (78.9) | 233 | (76.6) | 473 | (77.8) |
| Carcinoma | | | | | | |
| Extent of Disease, | | | | | | |
| n (%) | | | | | | |
| Metastatic | 284 | (93.4) | 290 | (95.4) | 574 | (94.4) |
| Recurrent/Persistent | 20 | (6.6) | 14 | (4.6) | 34 | (5.6) |
| Prior lines of therapy | | | | | | |
| for recurrent or | | | | | | |
| metastatic disease | | | | | | |
| 1 | 177 | (58.2) | 169 | (55.6) | 346 | (56.9) |
| >1 | 124 | (40.8) | 135 | (44.4) | 259 | (42.6) |
| Prior bevacizumab | | | | | | |
| use, n (%) | | | | | | |
| Yes | 149 | (49.0) | 147 | (48.4) | 296 | (48.7) |
| No | 155 | (51.0) | 157 | (51.6) | 312 | (51.3) |

The primary objective of the study was to compare overall survival (OS) for the patient population who have histology of squamous cell carcinoma (SCC) and then the overall population who have any eligible histology (SCC or adenocarcinoma/adenosquamous carcinoma [AC]), between cemiplimab and IC chemotherapy. Secondary objectives of the study performed among SCC patients and overall population (SCC and AC) were to compare progression free survival (PFS) in SCC, quality of life (QoL), objective response rate (ORR) in SCC, PFS and ORR in overall population and safety profiles between cemiplimab and IC chemotherapy. An interim analysis was scheduled when approximately 85% of events occurred among SCC patients.

Results: The 608 patients were randomized: median age: 51 years [range 22-87]; histology: 477 SCC, 131 AC; ECOG performance score: 0 [46.5%], 1 [53.5%]. Median cemiplimab exposure was 15 weeks (range: 1.4-100.7). At interim analysis, median OS for cemiplimab in the total population (n=304) versus IC chemotherapy (n=304) was 12.0 versus 8.5 months, respectively; hazard ratio (HR) for death 0.69; 95% confidence interval (CI) 0.56-0.84; p<0.001.

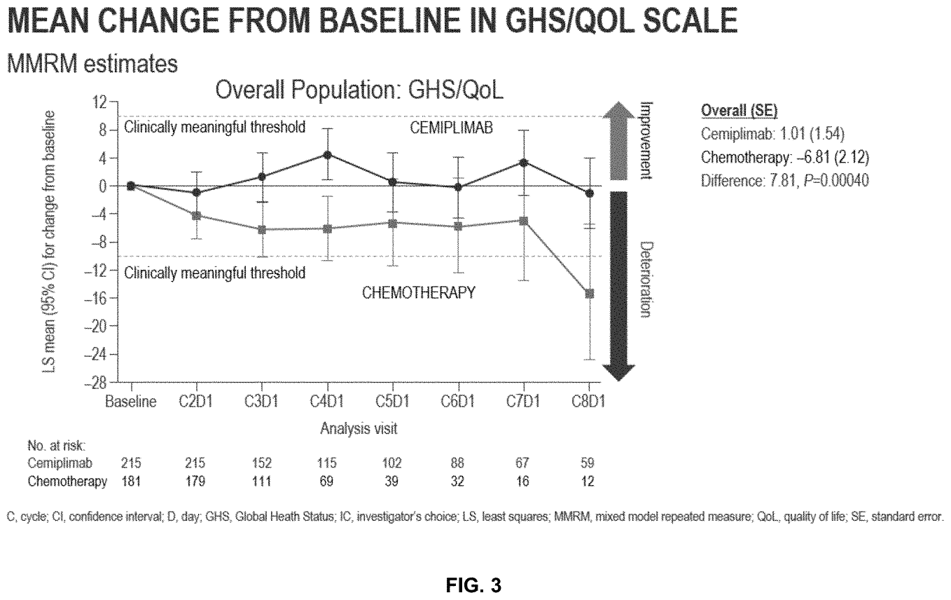

In the SCC population, OS was significantly longer in the cemiplimab arm than in the chemotherapy arm. Specifically, median OS for cemiplimab (n=239) versus IC chemotherapy (n=238) was 11.1 versus 8.8 months; HR for death 0.73; 95% CI 0.58-0.91; p=0.003. PFS in the SCC population was significantly superior in the cemiplimab arm than in the chemotherapy arm: hazard ratio=0.71 (95% CI: 0.58, 0.86); 1-sided p=0.00026. The estimated median PFS was 2.8 months in cemiplimab arm versus 2.9 months in chemotherapy arm. Overall mean change from baseline in Global Heath Status/Quality of Life (GHS/QoL) in SCC: The estimated difference in mean change from baseline (cemiplimab—chemotherapy) was 8.49 (95% CI: 3.77, 13.21); 1-sided p=0.00025; statistically significant and favors cemiplimab over chemotherapy. The mean change from baseline in GHS/QoL in the overall population is illustrated in FIG. 3. In the overall population, there was a nominally significant difference in favor of cemplimab over chemotherapy. Patients receiving cemiplimab improved or maintained GHS/QoL from baseline. Patients receiving chemotherapy generally showed deterioration in these scores.

Overall mean change from baseline in Physical Functioning in SCC: The estimated difference in mean change from baseline (cemiplimab—chemotherapy) was 8.35 (95% CI: 4.08, 12.62); 1-sided p=0.00008; statistically significant and favors cemiplimab over chemotherapy. ORR in SCC: ORR was significantly higher in the cemiplimab arm than in the chemotherapy arm: 17.6% (95% CI: 13.0-23.0) versus 6.7% (95% CI: 3.9-10.7), 1-sided p=0.00014.

In the AC population, median OS for cemiplimab (n=65) versus IC chemotherapy (n=66) was 13.3 versus 7.0 months; HR 0.56; 95% CI 0.36-0.85; p<0.005 (nominal p value). PFS, and ORR in overall and SCC populations, and mean change from baseline QoL in SCC, favored cemiplimab. PFS in AC: HR=0.91 (95% CI: 0.62, 1.34). The median PFS was 2.7 months in the cemiplimab arm versus 2.8 months in the chemotherapy arm. ORR in AC: ORR was 12.3% (95% CI: 5.5-22.8) in the cemiplimab arm versus 4.5% (95% CI: 0.9-12.7) in the chemotherapy arm.

The most common treatment emergent adverse events (AEs) of any grade for cemiplimab versus IC chemotherapy were anemia (25% versus 45%), nausea (18% versus 33%), and vomiting (16% versus 23%). Discontinuations due to AEs occurred in 8% of cemiplimab and 5% of IC chemotherapy patients. A safety summary is provided in Tables 14-15.

Table 14

| | n (%), unless stated | | | |
|---|---|---|---|---|
| | Cemiplimab | | Chemotherapy | |
| | (n = 300) | | (n = 290) | |
| | Median duration of exposure | | | |
| | (range), weeks | | | |
| | 15.2 (1.4-100.7) | | 10.1 (1.0-81.9) | |
| | Any | Grade | Any | Grade |
| | grade | 3-5 | grade | 3-5 |
| Treatment-emergent AEs, | | | | |
| regardless of attribution | | | | |
| Overall | 265 (88.3) | 135 (45.0) | 265 (91.4) | 155 (53.4) |
| Led to discontinuation | 26 (8.7) | 20 (6.7) | 15 (5.2) | 11 (3.8) |
| Led to death | 5 (1.7) | 5 (1.7) | 2 (0.7) | 2 (0.7) |
| Treatment-related AEs | | | | |
| Overall | 170 (56.7) | 44 (14.7) | 236 (81.4) | 117 (40.3) |
| Led to discontinuation | 17 (5.7) | 12 (4.0) | 10 (3.4) | 8 (2.8) |
| Led to death | 0 | 0 | 2 (0.7) | 2 (0.7) |
| Sponsor-identified immune-related AEs | | | | |
| Overall | 48 (16.0) | 18 (6.0) | 2 (0.7) | 2 (0.7) |
| Led to discontinuation | 15 (5.0) | 11 (3.7) | 2 (0.7) | 2 (0.7) |
| Led to death | 0 | 0 | 0 | 0 |

Safety was analyzed in all randomized patients who received any study treatment. AE, adverse events.

TABLE 15

| | Cemiplimab | | Chemotherapy | |
|---|---|---|---|---|
| | (n = 300) | | (n = 290) | |
| Treatment-emergent AEs in ≥15% | Any | Grade | Any | Grade |
| of patients in either arm, n (%) | grade | 3-5 | grade | 3-5 |
| Overall | 265 (88.3) | 135 (45.0) | 265 (91.4) | 155 (53.4) |
| Anaemia | 75 (25.0) | 36 (12.0) | 129 (44.5) | 78 (26.9) |
| Nausea | 55 (18.3) | 1 (0.3) | 97 (33.4) | 6 (2.1) |
| Fatigue | 50 (16.7) | 4 (1.3) | 45 (15.5) | 4 (1.4) |
| Vomiting | 48 (16.0) | 2 (0.7) | 68 (23.4) | 7 (2.4) |
| Decreased appetite | 45 (15.0) | 1 (0.3) | 46 (15.9) | 2 (0.7) |
| Constipation | 45 (15.0) | 0 | 59 (20.3) | 1 (0.3) |
| Pyrexia | 35 (11.7) | 1 (0.3) | 61 (21.0) | 0 |
| Asthenia | 33 (11.0) | 7 (2.3) | 44 (15.2) | 3 (1.0) |
| Neutropenia | 6 (2.0) | 3 (1.0) | 44 (15.2) | 26 (9.0) |

There were no immune-related AEs that are not well described for the PD-1/PD-L1 inhibitor class.

In the overall population with respect to OS, the efficacy analysis included 304 patients in both treatment arms. Median follow up (from randomization to data cutoff date) was 17.9 months in the cemiplimab arm versus 18.3 months in the chemotherapy arm. OS was significantly longer in the cemiplimab arm than in the chemotherapy arm: hazard ratio=0.69 (95% CI: 0.56, 0.84); 1-sided p=0.00011. The estimated median OS was 12.0 months in the cemiplimab arm versus 8.5 months in the chemotherapy arm. PFS in overall population: PFS was significantly superior in the cemiplimab arm as compared to the chemotherapy arm: hazard ratio=0.75 (95% CI: 0.63, 0.89); 1-sided p=0.00048. The estimated median PFS was 2.8 months in cemiplimab arm versus 2.9 months in chemotherapy arm. ORR in overall population: ORR was significantly higher in the cemiplimab arm than in the chemotherapy arm: 16.4% versus 6.3%, 1-sided p=0.00004.

Statistical Methods: The primary endpoint of OS was summarized using Kaplan-Meier survival curves and compared between the two treatment groups using a log-rank test stratified by randomization stratification factors of geographic region (North America vs Asia vs ROW) in SCC and in AC, and by histology (SCC vs AC) and geographic region in overall population. The hazard ratio with a two-sided 95% confidence interval was derived from a stratified Cox proportional hazards model with the same stratification factors used in the stratified log-rank test. PFS was analyzed using the same statistical method as described for the primary analysis of OS with regard to SCC, adenocarcinoma and overall population. ORR was analyzed using Cochran-Mantel-Haenszel test stratified by the same stratification factors used in OS analysis with regard to SCC, AC, and overall population. ORR and the corresponding 95% exact CI were calculated by Clopper-Pearson method for each treatment group.

QoL: Longitudinal change from baseline at each PRO assessment in the GHS/QoL and PF scales was analyzed using a mixed model with repeated measures (MMRM). Higher scores on these scales indicate better health status/function. Pairwise comparison of the overall adjusted mean estimates giving each visit equal weight, and the adjusted mean estimates at Cycle 2 were conducted for Cemiplimab versus IC chemotherapy. The model generated least squares (LS) mean estimates, standard errors, 95% CIs and p-values (where applicable) for mean changes from baseline on each PRO assessment.

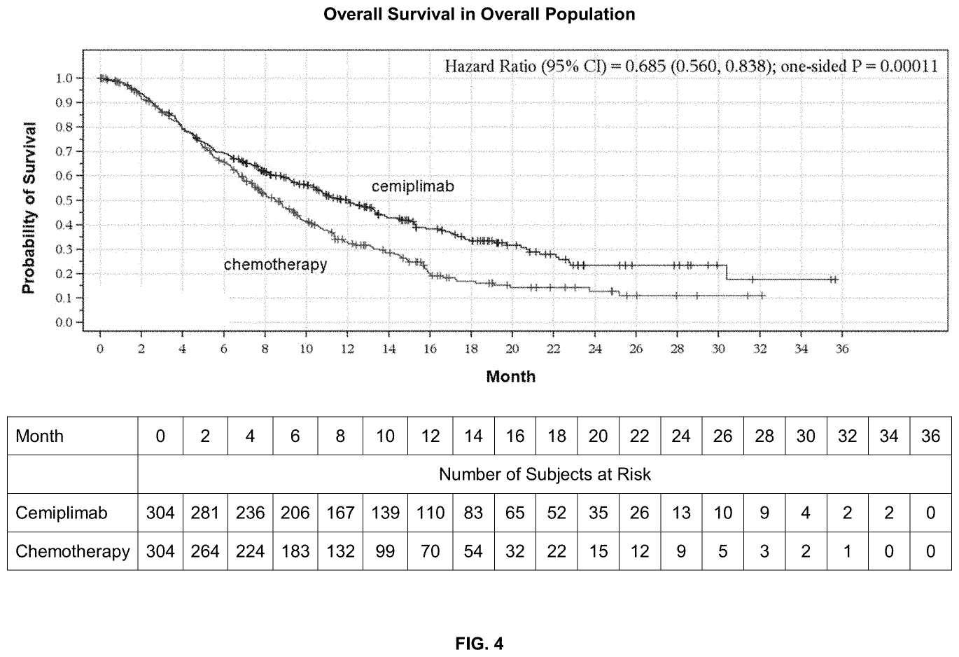

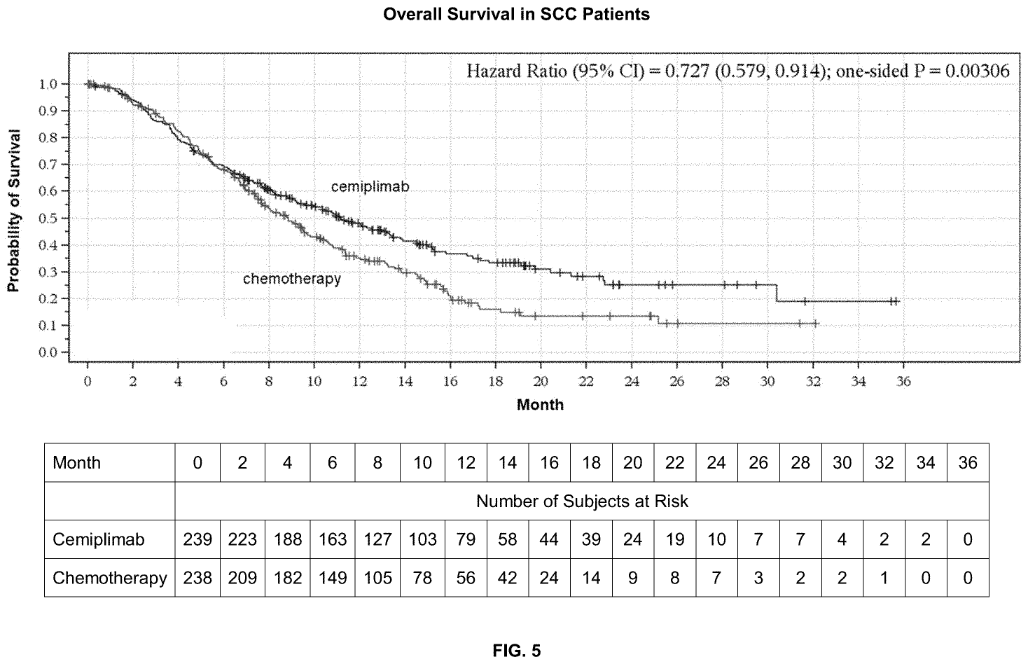

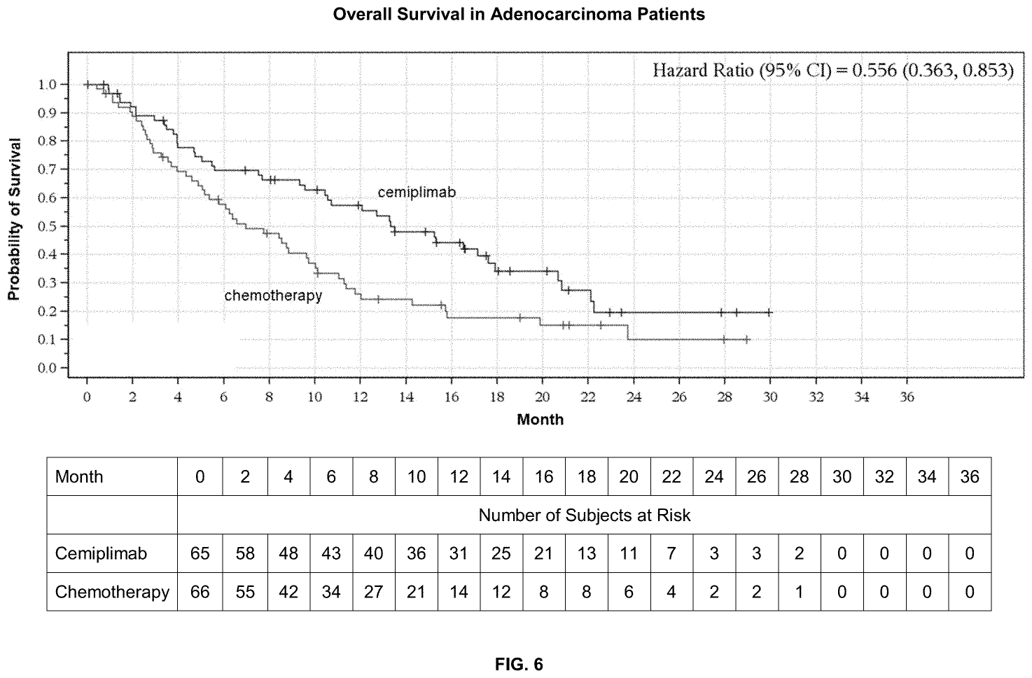

OS results are shown in Tables 16-19 and FIGS. 4-6.

TABLE 16
Overall Survival, Subgroup Analyses in Overall Population

| | Cemiplimab | Chemotherapy | Hazard Ratio |
|---|---|---|---|
| | (Events/Total) | (Events/Total) | (95% CI)* |
| Histology per IWRS | | | |
| SCC | 143/239 | 161/238 | 0.73 (0.58-0.91) |
| Non-SCC | 41/65 | 50/66 | 0.56 (0.36-0.85) |
| Geographic region- | | | |
| group 1 | | | |
| North America | 16/32 | 22/34 | 0.52 (0.27-1.00) |
| Asia | 54/83 | 54/83 | 0.65 (0.44-0.96) |
| Rest of World | 114/189 | 135/187 | 0.73 (0.57-0.94) |
| ECOG status per | | | |
| IWRS | | | |
| 0 | 73/146 | 88/146 | 0.59 (0.43-0.82) |
| 1 | 111/158 | 123/158 | 0.74 (0.57-0.96) |
| Prior bevacizumab | | | |
| use per IWRS | | | |
| Yes | 85/149 | 97/147 | 0.64 (0.48-0.86) |
| No | 99/155 | 114/157 | 0.76 (0.58-1.00) |
| No. of prior lines of | | | |
| systemic therapy for | | | |
| RIM disease | | | |
| 1 line | 143/177 | 152/169 | 0.74 (0.58-0.94) |
| >1 line | 109/124 | 117/135 | 0.72 (0.54-0.95) |

*Stratified by geographic region (North America vs Asia vs ROW per IWRS) and histology (SCC vs AC per IWRS) except for geographic region, histology subgroups (cemiplimab vs chemotherapy). Geographic region is stratified by histology (SCC versus AC per IWRS). Histology is stratified by geographic region (North America vs Asia vs ROW per IWRS) (cemiplimab vs chemotherapy). AC, adenocarcinoma or adenosquamous carcinoma; CI, confidence interval; ECOG, Eastern Cooperative Oncology Group; IWRS, interactive web response system; ROW, rest of world; R/M, recurrent or metastatic; SCC, squamous cell carcinoma.

TABLE 17
Overall Survival (OS) in Overall Population (full analysis set)

| | Cemiplimab | Chemotherapy |
|---|---|---|
| | (N = 304) | (N = 304) |
| Number of deaths, n (%) | 184 (60.5%) | 211 (69.4%) |
| Number of censored patients, n (%) | 120 (39.5%) | 93 (30.6%) |
| Median (95% CI), (months)[a] | 12.0 (10.3, 13.5) | 8.5 (7.5, 9.6) |
| Stratified log-rank test one-sided | 0.00011 | |
| p-value [b][c] | | |
| HR (95% CI) [b][d] | 0.685 (0.560, 0.838) | |
| Estimated Survival Probability, | | |
| % (95% CI)[a] | | |
| 6 months | 69.6 (64.0, 74.5) | 66.1 (60.3, 71.3) |
| 12 months | 50.2 (44.1, 56.0) | 33.2 (27.4, 39.0) |
| 18 months | 33.4 (27.2, 39.7) | 16.9 (11.9, 22.6) |
| 24 months | 23.6 (17.1, 30.6) | 12.8 (8.0, 18.8) |
| 30 months | 23.6 (17.1, 30.6) | 11.0 (6.1, 17.5) |
| 36 months | NE (NE, NE) | NE (NE, NE) |

[a]Based on Kaplan-Meier method.
[b]Stratified by geographic region (North America versus Asia versus ROW) and Histology (SCC versus adenocarcinoma) according to IWRS.
[c]One-sided p-value converted from stratified log-rank test two-sided p-value.
[d]Based on stratified proportional hazards model (cemiplimab vs chemotherapy).

TABLE 18
Overall Survival (OS) in SCC Population (full analysis set)

| | Cemiplimab | Chemotherapy |
|---|---|---|
| | (N = 239) | (N = 238) |
| Number of deaths, n (%) | 143 (59.8%) | 161 (67.6%) |
| Number of censored patients, n (%) | 96 (40.2%) | 77 (32.4%) |
| Median (95% CI), (months)[a] | 11.1 (9.2, 13.4) | 8.8 (7.6, 9.8) |
| Stratified log-rank test one- | 0.00306 | |
| sided p-value [b][c] | | |
| HR (95% CI) [b][d] | 0.727 (0.579, 0.914) | |
| Estimated Survival Probability, | | |
| % (95% CI)[a] | | |
| 6 months | 69.6 (63.3, 75.0) | 68.5 (61.9, 74.1) |
| 12 months | 48.2 (41.3, 54.7) | 35.3 (28.6, 42.1) |
| 18 months | 33.4 (26.3, 40.6) | 16.1 (10.4, 22.8) |
| 24 months | 25.3 (17.8, 33.5) | 13.6 (8.1, 20.4) |
| 30 months | 25.3 (17.8, 33.5) | 10.8 (5.2, 18.8) |
| 36 months | NE (NE, NE) | NE (NE, NE) |

[a]Based on Kaplan-Meier method.
[b]Stratified by geographic region (North America versus Asia versus ROW) according to IWRS. Significant threshold is set to 0.01508 using O Brien Fleming alpha spending function.
[c]One-sided p-value converted from stratified log-rank test two-sided p-value.
[d]Based on stratified proportional hazards model (cemiplimab vs chemotherapy).

TABLE 19
Overall Survival in Adenocarcinoma Patients (full analysis set)

| | Cemiplimab | Chemotherapy |
|---|---|---|
| | (N = 65) | (N = 66) |
| Number of deaths, n (%) | 41 (63.1%) | 50 (75.8%) |
| Number of censored patients, n (%) | 24 (36.9%) | 16 (24.2%) |
| Median (95% CI), (months)[a] | 13.3 (9.6, 17.6) | 7.0 (5.1, 9.7) |
| HR (95% CI) [b][c] | 0.556 (0.363, 0.853) | |
| Estimated Survival Probability, | | |
| % (95% CI)[a] | | |
| 6 months | 69.6 (56.5, 79.4) | 57.7 (44.4, 68.9) |
| 12 months | 57.4 (43.9, 68.7) | 26.0 (15.5, 37.8) |
| 18 months | 34.2 (21.5, 47.3) | 17.7 (8.9, 29.0) |
| 24 months | 19.5 (8.4, 34.1) | 10.1 (2.7, 23.3) |
| 30 months | NE (NE, NE) | NE (NE, NE) |
| 36 months | NE (NE, NE) | NE (NE, NE) |

[a]Based on Kaplan-Meier method.
[b]Stratified by geographic region (North America versus Asia versus ROW) according to IWRS.
[c]Based on stratified proportional hazards model (cemiplimab vs chemotherapy).

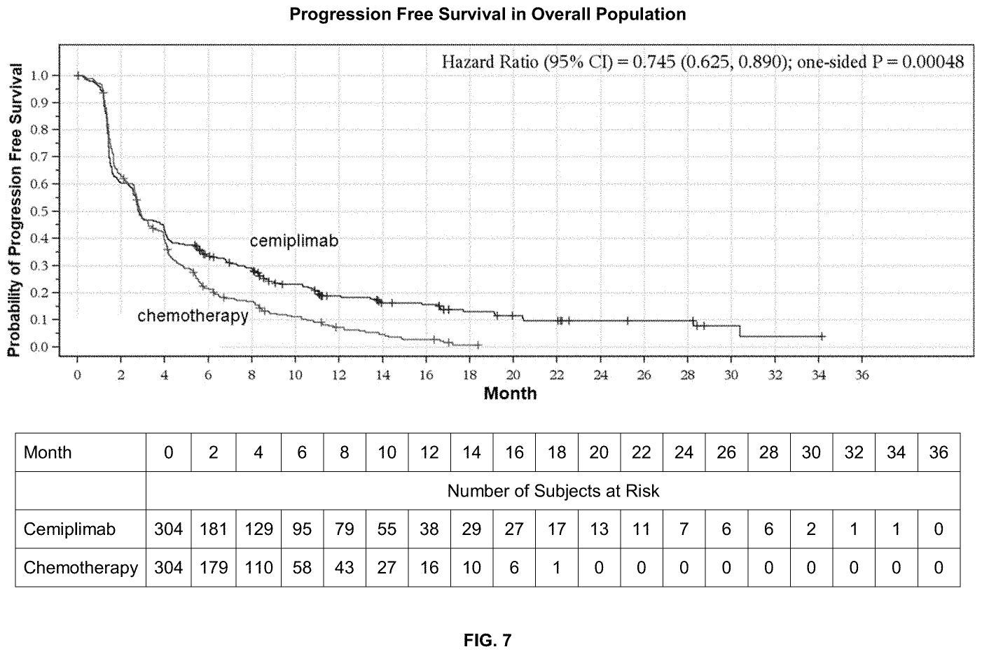

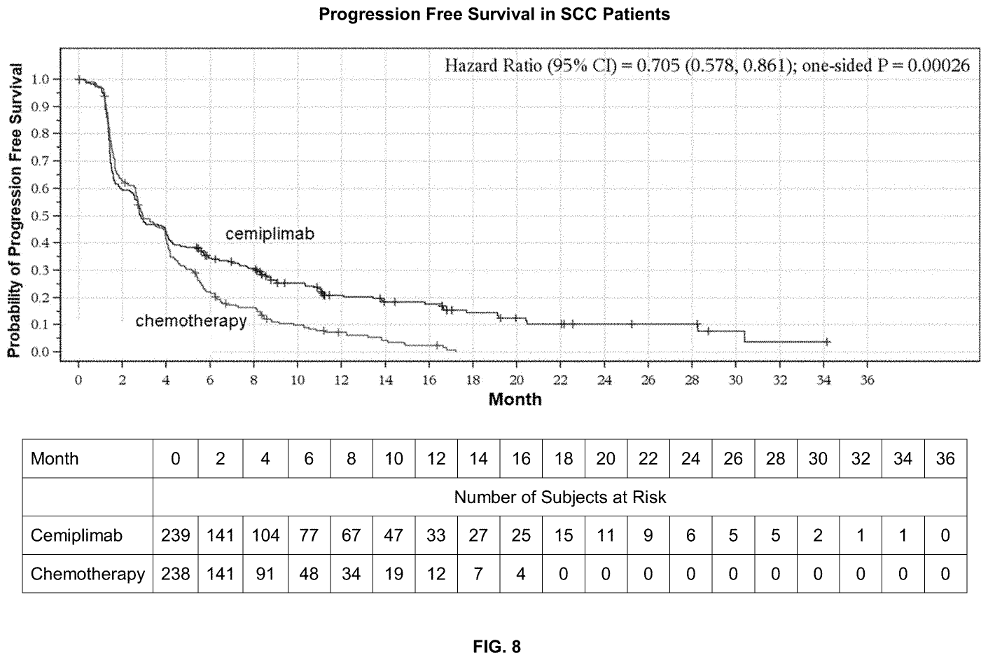

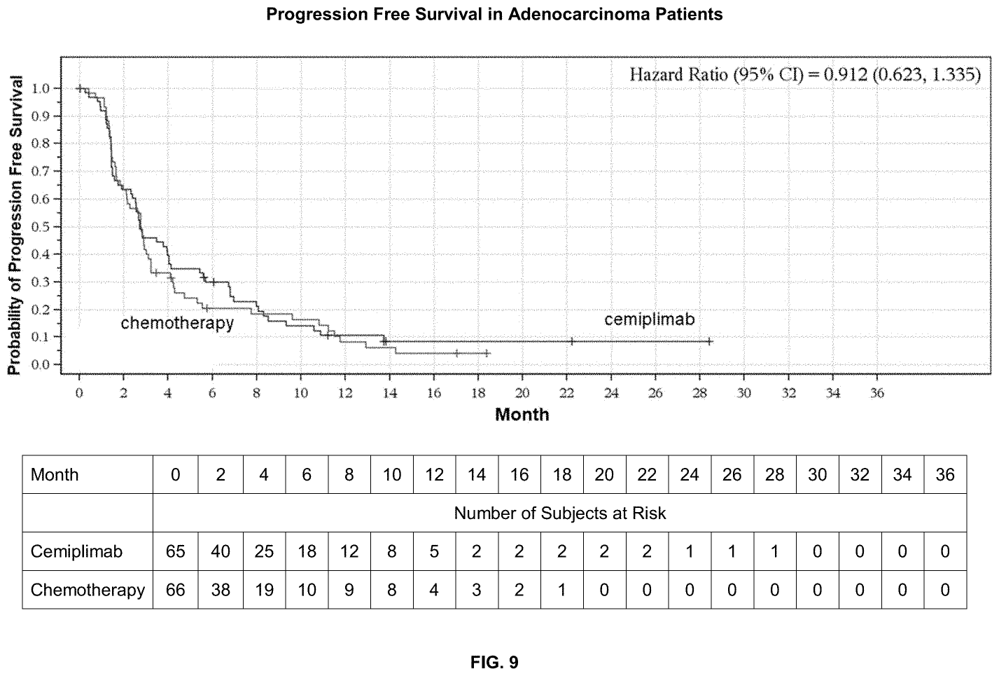

PFS results are shown in Tables 20-22 and FIGS. 7-9.

TABLE 20
Progression Free Survival in Overall Population (full analysis set)

| | Cemiplimab | Chemotherapy |
|---|---|---|
| | (N = 304) | (N = 304) |
| Number of events, n (%) | 253 (83.2%) | 269 (88.5%) |
| Progressive Disease, n (%) | 212 (69.7%) | 215 (70.7%) |
| Number of deaths, n (%) | 41(13.5%) | 54 (17.8%) |
| Number of censored patients, n (%) | 51(16.8%) | 35 (11.5%) |
| Median (95% CI), (months)[a] | 2.8 (2.6, 3.9) | 2.9 (2.7, 3.4) |
| Stratified log-rank test one-sided | 0.00048 | |
| p-value [b][c] | | |
| HR (95% CI) [b][d] | 0.745 (0.625, 0.890) | |
| Estimated Event-Free Probability, | | |
| % (95% CI)[a] | | |
| 6 months | 33.5 (28.2, 38.9) | 21.7 (17.1, 26.7) |
| 12 months | 18.8 (14.4, 23.6) | 7.3 (4.6, 11.0) |
| 18 months | 13.0 (9.0, 17.8) | 0.8 (0.1, 3.6) |
| 24 months | 9.7 (6.0, 14.6) | NE (NE, NE) |
| 30 months | 7.8 (3.8, 13.6) | NE (NE, NE) |
| 36 months | NE (NE, NE) | NE (NE, NE) |

[a]Based on Kaplan-Meier method.
[b]Stratified by geographic region (North America versus Asia versus ROW) and Histology (SCC versus adenocarcinoma) according to IWRS.
[c]One-sided p-value converted from stratified log-rank test two-sided p-value.
[d]Based on stratified proportional hazards model (cemiplimab vs chemotherapy).

TABLE 21
Progression Free Survival in SCC Patients (full analysis set)

| | Cemiplimab | Chemotherapy |
|---|---|---|
| | (N = 239) | (N = 238) |
| Number of events, n (%) | 197 (82.4%) | 214 (89.9%) |
| Progressive Disease, n (%) | 163 (68.2%) | 172 (72.3%) |
| Number of deaths, n (%) | 34 (14.2%) | 42 (17.6%) |
| Number of censored patients, n (%) | 42 (17.6%) | 24 (10.1%) |
| Median (95% CI), (months)[a] | 2.8 (2.6, 4.0) | 2.9 (2.7, 3.9) |
| Stratified log-rank test one-sided | 0.00026 | |
| p-value [b][c] | | |
| HR (95% CI) [b][d] | 0.705 (0.578, 0.861) | |
| Estimated Event-Free Probability, | | |
| % (95% CI)[a] | | |
| 6 months | 34.5 (28.5, 40.5) | 22.1 (16.9, 27.8) |
| 12 months | 21.0 (15.8, 26.6) | 7.3 (4.2, 11.4) |
| 18 months | 14.5 (9.7, 20.1) | 0.0 (NE, NE) |
| 24 months | 10.3 (5.9, 16.0) | 0.0 (NE, NE) |
| 30 months | 7.7 (3.2, 14.8) | 0.0 (NE, NE) |
| 36 months | NE (NE, NE) | 0.0 (NE, NE) |

[a]Based on Kaplan-Meier method.
[b]Stratified by geographic region (North America versus Asia versus ROW) according to IWRS.
[c]One-sided p-value converted from stratified log-rank test two-sided p-value.
[d]Based on stratified proportional hazards model (cemiplimab vs chemotherapy).

TABLE 22
Progression Free Survival in Adenocarcinoma Patients (full analysis set)

| | Cemiplimab | Chemotherapy |
|---|---|---|
| | (N = 65) | (N = 66) |
| Number of events, n (%) | 56 (86.2%) | 55 (83.3%) |
| Progressive Disease, n (%) | 49 (75.4%) | 43 (65.2%) |
| Number of deaths, n (%) | 7 (10.8%) | 12 (18.2%) |
| Number of censored patients, n (%) | 9 (13.8%) | 11 (16.7%) |
| Median (95% CI), (months)[a] | 2.7 (2.3, 4.0) | 2.8 (2.0, 3.2) |
| HR (95% CI) [b][c] | 0.912 (0.623, 1.335) | |
| Estimated Event-Free Probability, | | |
| % (95% CI)[a] | | |
| 6 months | 30.1 (19.3, 41.6) | 20.4 (11.2, 31.7) |
| 12 months | 10.6 (4.4, 20.0) | 8.2 (2.7, 17.6) |
| 18 months | 8.5 (3.0, 17.6) | 4.1 (0.8, 12.2) |
| 24 months | 8.5 (3.0, 17.6) | NE (NE, NE) |
| 30 months | NE (NE, NE) | NE (NE, NE) |
| 36 months | NE (NE, NE) | NE (NE, NE) |

[a]Based on Kaplan-Meier method.
[b]Stratified by geographic region (North America versus Asia versus ROW) according to IWRS.
[c]Based on stratified proportional hazards model (cemiplimab vs chemotherapy).

ORR results are shown in Tables 23-25.

TABLE 23
Objective Response Rate in Overall Population (full analysis set)

| | Cemiplimab | Chemotherapy |
|---|---|---|
| | (N = 304) | (N = 304) |
| Best Overall Tumor Response, n (%) | | |
| Complete Response (CR) [a] | 10 (3.3) | 3 (1.0) |
| Partial Response (PR) [a] | 40 (13.2) | 16 (5.3) |
| Stable Disease (SD) [b] | 125 (41.1) | 148 (48.7) |
| Progressive Disease (PD) | 105 (34.5) | 88 (28.9) |
| Not Evaluable (NE) | 24 (7.9) | 49 (16.1) |
| Response | | |
| Objective Response Rate (ORR:CR + PR) | 50 (16.4%) | 19 (6.3%) |
| 95% CI for ORR [c] | (12.5%, 21.1%) | (3.8%, 9.6%) |
| Stratified CMH test one-sided p-value [d] | 0.00004 | |
| Odds ratio (95% CI) [d] | 2.984 (1.707, 5.215) | |
| Median DOR, months (95% CI)^{e} | 16.4 (12.4, NE) | 6.9 (5.1, 7.7) |
| Median observed time to response, months (range) | 2.7 (1.2-11.4) | 1.6 (1.2-9.0) |

[a]CR/PR must be confirmed by repeated assessments no less than 4 weeks apart
[b]SD criteria must be met at least once for a minimum duration of 4 weeks after first dose date
[c]Clopper-Person exact confidence interval.
[d]One-sided p-value and odds ratio using geographic region and histology stratified Cochran-Mantel-Haenszel (CMH) test. Due to the low response rate in the Chemotherapy arm, the results from CMH test should be interpreted with caution.

TABLE 24
Objective Response Rate in SCC Patients (full analysis set)

| | Cemiplimab | Chemotherapy |
|---|---|---|
| | (N = 239) | (N = 238) |
| Best Overall Tumor Response, | | |
| n (%) | | |
| Complete Response (CR) [a] | 7 (2.9%) | 2 (0.8%) |
| Partial Response (PR) [a] | 35 (14.6%) | 14 (5.9%) |
| Stable Disease (SD) [b] | 93 (38.9%) | 116 (48.7%) |
| Progressive Disease (PD) | 86 (36.0%) | 71 (29.8%) |
| Not Evaluable (NE) | 18 (7.5%) | 35 (14.7%) |
| Response | | |
| Objective Response Rate | 42 (17.6%) | 16 (6.7%) |
| (ORR:CR + PR) | | |
| 95% CI for ORR [c] | (13.0%, 23.0%) | (3.9%, 10.7%) |
| Stratified CMH test one-sided | 0.00014 | |
| p-value [d] | | |
| Odds ratio (95% CI) [d] | 3.002 (1.629, 5.530) | |

[a]CR/PR must be confirmed by repeated assessments no less than 4 weeks apart
[b]SD criteria must be met at least once for a minimum duration of 4 weeks after first dose date
[c]Clopper-Person exact confidence interval.
[d]One-sided p-value and odds ratio using geographic region stratified Cochran-Mantel-Haenszel test. Due to the low response rate in the Chemotherapy arm, the results from CMH test should be interpreted with caution.

TABLE 25
Objective Response Rate in Adenocarcinoma Patients (full analysis set)

| | Cemiplimab | Chemotherapy |
|---|---|---|
| | (N = 65) | (N = 66) |
| Best Overall Tumor Response, n (%) | | |
| Complete Response (CR) [a] | 3 (4.6%) | 1 (1.5%) |
| Partial Response (PR) [a] | 5 (7.7%) | 2 (3.0%) |
| Stable Disease (SD) [b] | 32 (49.2%) | 32 (48.5%) |
| Progressive Disease (PD) | 19 (29.2%) | 17 (25.8%) |
| Not Evaluable (NE) | 6 (9.2%) | 14 (21.2%) |
| Response | | |
| Objective Response Rate (ORR: | 8 (12.3%) | 3 (4.5%) |
| CR + PR) | | |
| 95% CI for ORR [c] | (5.5%, 22.8%) | (0.9%, 12.7%) |
| Odds ratio (95% CI) [d] | 2.894 (0.732, 11.445) | |

[a]CR/PR must be confirmed by repeated assessments no less than 4 weeks apart
[b]SD criteria must be met at least once for a minimum duration of 4 weeks after first dose date
[c]Clopper-Person exact confidence interval.
[d]Odds ratio using geographic region stratified Cochran-Mantel-Haenszel test. Due to the low response rate in the Chemotherapy arm, the results from CMH test should be interpreted with caution.

QoL and Physical Functioning results are shown in Tables 26-29.

TABLE 26
Least Square Mean Estimates of Changes from Baseline
of EORTC QLQ-C30 GHS/QoL in SCC Patients (full analysis set)

| | Cemiplimab | | Chemotherapy | | Cemiplimab versus Chemotherapy | | |
|---|---|---|---|---|---|---|---|
| | Estimated | | Estimated | | Estimated | | p-value |
| Visit | Mean | 95% Cl | Mean | 95% Cl | Mean | 95% Cl | (one-sided) |
| C2D1 | −2.02 | (−5.328, | −6.16 | (−9.710, | 4.14 | (−0.213, | 0.03113 |
| | | 1.298) | | −2.603) | | 8.496) | |
| C3D1 | 1.18 | (−2.668 | −8.23 | (−12.554 | 9.41 | (4.074, | |
| | | 5.018), | | −3.908), | | 14.738) | |
| C4D1 | 5.15 | (1.037, | −7.21 | (−12.240, | 12.36 | (6.286, | |
| | | 9.264) | | −2.188) | | 18.442) | |
| C5D1 | 0.85 | (−3.785 | −6.10 | (−12.707, | 6.94 | (−0.787, | |
| | | 5.482), | | 0.515) | | 14.677) | |
| C6D1 | −0.17 | (−4.920, | −7.01 | (−14.179, | 6.83 | (−1.445, | |
| | | 4.574) | | 0.168) | | 15.110) | |
| C7D1 | 2.09 | (−3.264, | −9.16 | (−19.270, | 11.25 | (0.011, | |
| | | 7.443) | | 0.949) | | 22.487) | |
| Overall | 1.18 | (−1.981, | −7.31 | (−11.499, | 8.49 | (3.771, | 0.00025 |
| Across | | 4.340) | | −3.122) | | 13.209) | |
| Cycles | | | | | | | |

The dependent variable for the Mixed Model Repeated Measures (MMRM) model is change from baseline of PRO scores and the predictors are the corresponding baseline PRO scores, treatment, visit, geographic region, treatment by visit, and baseline PRO scores by visit interactions. Scheduled visits with less than 10 patients in either arm, unscheduled visits, and off-treatment visits are not included in the analysis.

TABLE 27
Least Square Mean Estimates of Changes from Baseline
of EORTC QLQ-C30 Physical Functioning in SCC Patients-FAS

| | Cemiplimab | | Chemotherapy | | Cemiplimab versus Chemotherapy | | |
|---|---|---|---|---|---|---|---|
| | Estimated | | Estimated | | Estimated | | p-value |
| Visit | Mean | 95% Cl | Mean | 95% Cl | Mean | 95% Cl | (one-sided) |
| C2D1 | −1.82 | (−4.568, | −5.92 | (−8.856, | 4.09 | (0.542, | 0.01201 |
| | | 0.919) | | −2.978) | | 7.644) | |
| C3D1 | 2.10 | (−1.003 | −5.23 | (−8.683, | 7.33 | (3.118, | |
| | | 5.203), | | −1.785) | | 11.550) | |
| C4D1 | 5.21 | (1.982, | −5.19 | (−9.065, | 10.41 | (5.750, | |
| | | 8.442) | | −1.323) | | 15.062) | |
| C5D1 | −0.17 | (−3.779 | −7.03 | (−11.943 | 6.86 | (1.076, | |
| | | 3.446), | | −2.111), | | 12.644) | |
| C6D1 | 0.82 | (−3.356, | −6.30 | (−12.428, | 7.11 | (−0.034, | |
| | | 4.988) | | −0.163) | | 14.256) | |
| C7D1 | −0.07 | (−5.105 | −14.36 | (−23.631 | 14.30 | (3.917, | |
| | | 4.968), | | −5.095), | | 24.673) | |
| Overall | 1.01 | (−1.859, | −7.34 | (−11.052, | 8.35 | (4.081, | 0.00008 |
| Across | | 3.882) | | −3.625) | | 12.619) | |
| Cycles | | | | | | | |

The dependent variable for the Mixed Model Repeated Measures (MMRM) mode is change from baseline of PRO scores and the predictors are the corresponding baseline PRO scores, treatment, visit, geographic region, treatment by visit, and baseline PRO scores by visit interactions. Scheduled visits with less than 10 patients in either arm, unscheduled visits, and off-treatment visits are not included in the analysis.

TABLE 28
Least Square Mean Estimates of Changes from Baseline
of EORTC QLQ-C30 GHS/QoL in Overall Population-FAS

| | Cemiplimab | | Chemotherapy | | Cemiplimab versus Chemotherapy | | |
|---|---|---|---|---|---|---|---|
| | Estimated | | Estimated | | Estimated | | p-value |
| Visit | Mean | 95% Cl | Mean | 95% Cl | Mean | 95% Cl | (one-sided) |
| C2D1 | −1.03 | (−4.102, | −4.28 | (−7.561, | 3.25 | (−0.540, | 0.04632 |
| | | 2.036) | | −0.996) | | 7.031) | |
| C3D1 | 1.19 | (−2.321 | −6.22 | (−10.204 | 7.41 | (2.719, | |
| | | 4.702), | | −2.245), | | 12.111) | |
| C4D1 | 4.47 | (0.784, | −6.09 | (−10.594, | 10.55 | (5.295, | |
| | | 8.148) | | −1.579) | | 15.810) | |
| C5D1 | 0.48 | (−3.727, | −5.18 | (−11.356, | 5.66 | (−1.371, | |
| | | 4.691) | | 0.998) | | 12.693) | |
| C6D1 | −0.29 | (−4.647, | −5.73 | (−12.463, | 5.44 | (−2.167, | |
| | | 4.074) | | 1.002) | | 13.055) | |
| C7D1 | 3.32 | (−1.351, | −4.98 | (−13.525, | 8.30 | (−1.142, | |
| | | 7.989) | | 3.568) | | 17.737) | |
| C8D1 | −1.09 | (−6.145, | −15.17 | (−24.904, | 14.08 | (3.415, | |
| | | 3.964) | | −5.440) | | 24.748) | |
| Overall | 1.01 | (−2.033, | −6.81 | (−10.977, | 7.81 | (3.295, | 0.00040 |
| Across | | 4.047) | | −2.637) | | 12.333) | |
| Cycles | | | | | | | |

The dependent variable for the Mixed Model Repeated Measures (MMRM) model is change from baseline of PRO scores and the predictors are the corresponding baseline PRO scores, treatment, visit, geographic region, histology, treatment by visit, and baseline PRO scores by visit interactions. Scheduled visits with less than 10 patients in either arm, unscheduled visits, and off-treatment visits are not included in the analysis.

TABLE 29
Least Square Mean Estimates of Changes from Baseline
of EORTC QLQ-C30 Physical Functioning in Overall Population-FAS

| | Cemiplimab | | Chemotherapy | | Cemiplimab versus Chemotherapy | | |
|---|---|---|---|---|---|---|---|
| | Estimated | | Estimated | | Estimated | | p-value |
| Visit | Mean | 95% Cl | Mean | 95% Cl | Mean | 95% Cl | (one-sided) |
| C2D1 | −2.41 | (−4.979, | −6.57 | (−9.301, | 4 16 | (1.085 | 0.00406 |
| | | 0.158) | | −3.830) | | 7.225), | |
| C3D1 | 0.49 | (−2.431, | −5.53 | (−8.804, | 6.02 | (2.220, | |
| | | 3.417) | | −2.252) | | 9.821) | |
| C4D1 | 2.38 | (−0.776, | −6.94 | (−10.745, | 932 | (4.891, | |
| | | 5.530) | | −3.140) | | 13.747) | |
| C5D1 | −1.27 | (−4.578, | −8.00 | (−12.555, | 6.74 | (1.555, | |
| | | 2.043) | | −3.455) | | 11.920) | |
| C6D1 | −1.34 | (−5.194, | −8.28 | (−13.964, | 6.93 | (0.440, | |
| | | 2.508) | | −2.591) | | 13.430) | |
| C7D1 | −0.10 | (−4.414, | −11.77 | (−19.459 | 11.66 | (3.094, | |
| | | 4.205) | | −4.074), | | 20.230) | |
| C8D1 | −0.89 | (−5.153, | −13.85 | (−21.720 | 12.96 | (4.261, | |
| | | 3.368) | | −5.978), | | 21.652) | |
| Overall | −0.45 | (−3.197 | −8.70 | (−12.300, | 8.26 | (4.291, | 0.00003 |
| Across | | 2.298), | | −5.110) | | 12.219) | |
| Cycles | | | | | | | |

The dependent variable for the Mixed Model Repeated Measures (MMRM) model is change from baseline of PRO scores and the predictors are the corresponding baseline PRO scores, treatment, visit, geographic region, histology, treatment by visit, and baseline PRO scores by visit interactions. Scheduled visits with less than 10 patients in either arm, unscheduled visits, and off-treatment visits are not included in the analysis.

At baseline and Day 1 of each treatment cycle (up to 16), patients were administered the EORTC QLQ-030. Mixed-effects repeated measures models estimated least squares (LS) mean change from baseline across all scales. Responder analyses determined the proportions with clinically meaningful (using 10-point threshold) improvement or deterioration, or stability on QLQ-030. Results are reported for SCC and overall population per statistical hierarchy; post-hoc analyses are presented for the AC cohort.

Baseline scores showed moderate to high functioning and low to moderate symptom burden with minimal differences across treatment groups. Significant differences in LS mean changes in favor of cemiplimab were observed for QLQ-C30 GHS/QoL, physical functioning scales and other functioning and symptom scales (Table 30). Estimated treatment effect for role functioning, pain and appetite loss exceeded the clinically meaningful threshold. More patients receiving cemiplimab experienced clinically meaningful improvement/maintenance across GHS/QoL, functioning and most symptom scales. In patients with recurrent or metastatic cervical cancer, cemiplimab provided significant benefit versus chemotherapy in maintaining/improving GHS/QoL, physical functioning and most symptoms.

TABLE 30
Overall difference in LS mean change from baseline (95% CI)^{a}

| | Overall | SCC | AC |
|---|---|---|---|
| GHS/QoL | 7.8 (3.3; 12.3)* | 8.5 (3.8; 13.2)^{†} | 2.0 (−5.4; 9.3) |
| Physical | 8.3 (4.3; 12.2)* | 8.4 (4.1; 12.6)^{†} | 2.6 (−4.3; 9.5) |
| functioning | | | |
| Role | 13.1 (8.3; 17.9)^{‡} | 11.4 (6.3; 16.6)^{‡} | 8.3 (−0.0; 16.6) |
| functioning | | | |
| Fatigue | −9.1 (−14.1; −4.0)^{‡} | −8.6 (−14.0; −3.3)^{‡} | −5.9 (−13.9; 2.2) |
| Pain | −10.2 (−16.2; −4.3)^{‡} | −10.4 (−16.9; −3.8)^{‡} | −7.6 (−17.1; 1.9) |
| Appetite loss | −11.3 (−16.6; −6.0)^{‡} | −10.9 (−16.6; −5.2)^{‡} | −5.9 (−19.8; 2.4) |

*one-sided nominal P < 0.001;
^{†}one-sided P < 0.001;
^{‡}two-sided nominal P < .05.
aCemiplimab minus chemotherapy; positive numbers favor cemiplimab; for symptom scale, negative numbers favor cemiplimab. CI, confidence interval.

In the overall population, those treated with cemiplimab (n=304) experienced significant improvements in OS, PFS, and ORR, compared to chemotherapy (n=304), including: (i) 31% reduction in the risk of death ([HR: 0.69; 95% CI: 0.56-0.84; one-sided p=0.0011); (ii) 25% reduction in the risk of disease progression (HR=0.75; 95% CI: 0.63-0.89; one-sided p=0.00048); and (iii) 16% ORR (50 patients; 95% CI: 13-21%; one-sided p=0.00004) compared to 6% for chemotherapy (19 patients), wherein median duration of response was 16 months for cemiplimab (95% CI: 12 months to not yet evaluable) and 7 months for chemotherapy (95% CI: 5-8 months), per Kaplan-Meier estimates.

In this study, 78% of patients had advanced cervical cancer that was classified as SCC. In this subpopulation, significant improvements were also seen with cemiplimab (n=239), compared to chemotherapy (n=238), including: (i) 27% reduction in the risk of death (HR: 0.73; 95% CI: 0.58-0.91; one-sided p=0.00306); (ii) 29% reduction in the risk of disease progression (HR=0.71; 95% CI: 0.58-0.86; one-sided p=0.00026); and (iii) 18% ORR (42 patients; 95% CI: 13-23%) compared to 7% for chemotherapy (16 patients; 95% CI: 6-23%).

While assessment of the adenocarcinoma was not a pre-specified endpoint, a post-hoc analysis demonstrated the following outcomes for cemiplimab-treated patients (n=65) compared to chemotherapy (n=66), including: (i) 44% reduction in the risk of death (HR: 0.56; 95% CI: 0.36-0.85; nominal one-sided p<0.005); (ii) 9% reduction in the risk of disease progression (HR=0.91; 95% CI: 0.62-1.34); and (iii) 12% ORR (8 patients; 95% CI: 6-23%) compared to 5% for chemotherapy (3 patients; 95% CI: 1-13%).

Additionally, cemiplimab-treated patients were able to generally improve or maintain their baseline GHS/QOL, while those treated with chemotherapy experienced a deterioration that became clinically meaningful by cycle 8, per the EORTC QLQ-C30 (overall estimated results [standard error]: improvement of 1.01 [1.54] for cemiplimab, worsening of −6.81 [2.12] for chemotherapy; difference: 7.81; one-sided nominal P=0.00040).

No new cemiplimab safety signals were observed. Safety was assessed in patients who received at least 1 dose of study treatment: 300 patients in the cemiplimab group (median duration of exposure: 15 weeks; range: 1-101 weeks) and 290 patients in the chemotherapy group (median duration of exposure: 10 weeks; range: 1-82 weeks). AEs were observed in 88% of cemiplimab patients and 91% of chemotherapy patients, with those occurring in 15% or more cemiplimab patients being anemia (25% cemiplimab, 45% chemotherapy), nausea (18% cemiplimab, 33% chemotherapy), fatigue (17% cemiplimab, 16% chemotherapy), vomiting (16% cemiplimab, 23% chemotherapy), decreased appetite (15% cemiplimab, 16% chemotherapy) and constipation (15% cemiplimab, 20% chemotherapy). Grade 3 or higher AEs occurred in 45% of cemiplimab patients and 53% of chemotherapy patients. Among AEs in 15% or more patients, Grade 3 or higher AEs that occurred more often in the cemiplimab group included asthenia (2% cemiplimab, 1% chemotherapy) and pyrexia (less than 1% cemiplimab, 0% chemotherapy). Immune-related AEs were observed in 16% of cemiplimab patients and less than 1% of chemotherapy patients, with 6% and less than 1% being Grade 3 or higher, respectively. Discontinuations due to AEs occurred in 9% of cemiplimab patients and 5% of chemotherapy patients.

Conclusions: Cemiplimab monotherapy produced a statistically significant improvement in OS, PFS and ORR over investigator's choice chemotherapy in the overall population and in both SCC and AC subpopulations of patients with recurrent or metastatic cervical cancer that has progressed after platinum-containing chemotherapy in the 2L setting. In patients with histology of squamous cell carcinoma, the statistically significant difference in overall mean change from baseline in Global Heath Status/Quality of Life and Physical Functioning favored cemiplimab over investigator's choice chemotherapy. Additionally, the overall mean change from baseline GHS/QoL favored cemiplimab in the overall population (nominal P<0.001) and SCC patients (P<0.001). The toxicity profile of cemiplimab compared favorably with that of chemotherapy (fewer TEAEs of any grade and Grade 3), and was consistent with the known safety profile of cemiplimab.

In summary, this study, which enrolled patients regardless of PD-L1 expression status, shows that cemiplimab is a highly effective treatment for women with second-line advanced cervical cancer who face a poor prognosis and limited treatment options. Cemiplimab demonstrated a significant improvement in OS in women with advanced cervical cancer after progression on chemotherapy, reducing the risk of death by 31% compared to chemotherapy in the overall population. Moreover, cemiplimab resulted in significantly longer OS than single agent chemotherapy for patients with recurrent or metastatic cervical cancer after 1L platinum-based therapy regardless of PD-L1 status or histology (SCC, AC); and no new safety signals were observed. Cemiplimab is the first immunotherapy to demonstrate OS benefit in recurrent or metastatic cervical cancer and provides a new standard of care treatment option for this poor prognosis population.

### Example 5: Results of Phase 3 Recurrent or Metastatic Cervical Cancer Trial; Subgroup Efficacy Analysis of Cemiplimab Versus Investigator's Choice Chemotherapy

This example provides results obtained from the clinical trial described in Example 2, which is an open-label, randomized (1:1), multi-centre, Phase 3 clinical trial of anti-PD-1 cemiplimab vs investigator's choice (IC) single agent chemotherapy in recurrent or metastatic cervical cancer that has progressed after first-line platinum-based treatment. Single-agent chemotherapy selected by the investigator included gemcitabine, pemetrexed, vinorelbine, topotecan, or irinotecan. Adult females (age years) were enrolled regardless of PD-L1 expression and received cemiplimab 350 mg intravenously every 3 weeks or IC chemotherapy for up to 96 weeks; and were stratified by histology (squamous cell carcinoma [SCC]/adenocarcinoma or adenosquamous [AC]), geographic region (North America/Asia/rest of world), prior bevacizumab, and ECOG performance status (0/1). Primary endpoint was OS. Additional endpoints included progression-free survival (PFS), objective response rate (ORR), duration of response, quality of life, and safety.

A total of 608 patients were randomized: 304 to cemiplimab and 304 to IC chemotherapy (gemcitabine, n=121; premetrexed, n=111; vinorelbine, n=32; topotecan, n=21; irinotecan, n=19) across geographic regions and histologies. Median duration of study follow-up (range) was 4.8 months (0.0-25.9) for the overall population. At second interim analysis, the trial was stopped early for efficacy. OS, PFS and ORR (Table 31) demonstrated improvements with cemiplimab versus each IC chemotherapy treatment similar to those observed with cemiplimab versus pooled IC chemotherapy. Overall, improvements in OS, PFS, and ORR with cemiplimab trended consistently with the results for the overall population regardless of IC chemotherapy drug.

TABLE 31

| Cemiplimab | | | | | |
|---|---|---|---|---|---|
| vs | Investigator choice chemotherapy prior to randomisation | | | | |
| Individual IC | Pemetrexed | Topotecan | Irinotecan | Gemcitabine | Vinorelbine |
| chemotherapy | (n = 119 vs 111) | (n = 20 vs 21) | (n = 26 vs 19) | (n = 108 vs 121) | (n = 31 vs 32) |
| Median OS | 12.5 (7.5-15.3) | 7.0 (3.9-15.0) | 15.8 (11.2-NE) | 10.7 (9.2-13.3) | 10.3 (3.4, 22.8) |
| | vs | vs | vs | vs | vs |
| | 7.7 (6.4-9.8) | 6.5 (4.4-8.8) | 11.8 (6.9-14.9) | 9.0 (7.0-10.6) | 7.6 (5.2, 13.2) |
| | HR; 0.71 | HR; 0.78 | HR; 0.69 | HR; 0.76 | HR; 0.77 |
| | (0.52-0.98) | (0.31-1.96) | (0.28-1.70) | (0.54-1.06) | (0.40-1.48) |
| Median PFS | 3.0 (2.3-4.2) | 2.2 (1.3-4.1) | 6.9 (1.9-13.8) | 2.8 (2.6-4.3) | 1.5 (1.4-2.7) |
| | vs | vs | vs | vs | vs |
| | 2.9 (2.6-3.7) | 2.3 (1.7-5.4) | 4.2 (1.6-8.3) | 2.8 (2.1-3.9) | 2.8 (1.5-4.1) |
| | HR; 0.70 | HR; 0.90 | HR; 0.77 | HR; 0.73 | HR; 1.21 |
| | (0.52-0.94) | (0.43-1.90) | (0.34-1.72) | (0.54-0.97) | (0.70-2.09) |
| ORR | 16.0 | 15.0 | 23.1 | 17.6 | 9.7 |
| | (9.9-23.8) | (3.2-37.9) | (9.0-43.6) | (10.9-26.1) | (2.0-25.8) |
| | vs | vs | vs | vs | vs |
| | 6.3 | 4.8 | 15.8 | 5.0 | 6.3 |
| | (2.6-12.6) | (0.1-23.8) | (3.4-39.6) | (1.8-10.5) | (0.8-20.8) |

Data shown by individual investigator choice of chemotherapy selected prior to randomization in cemiplimab
versus chemotherapy arms. OS and PFS data shown as months, 95% CI; ORR data shown as %, 95% CI.
CI, confidence interval; HR, hazard ratio.

### REFERENCES

[1] F. Bray, J. Ferlay, I. Soerjomataram, R. L. Siegel, L. A. Torre, A. Jemal, Global cancer statistics 2018: GLOBOCAN estimates of incidence and mortality worldwide for 36 cancers in 185 countries, CA Cancer J Clin 68 (2018) 394-424.
[2] R. Burk, Z. Chen, C. Sailer, T. C. G. A. R. Network, Integrated genomic and molecular characterization of cervical cancer, Nature 543 (2017) 378-384.
[3] C. Marth, F. Landoni, S. Mahner, M. McCormack, A. Gonzalez-Martin, N. Colombo, E. G. Committee, Cervical cancer: ESMO Clinical Practice Guidelines for diagnosis, treatment and follow-up, Ann Oncol 28 (2017) iv72-iv83.
[4] K. S. Tewari, M. W. Sill, H. J. Long III, R. T. Penson, H. Huang, L. M. Ramondetta, L. M. Landrum, A. Oaknin, T. J. Reid, M. M. Leitao, Improved survival with bevacizumab in advanced cervical cancer, N Engl J Med 370 (2014) 734-743.
[5] D. Lorusso, G. Ferrandina, S. Pignata, M. Ludovisi, R. Vigano, S. Scalone, P. Scollo, E. Breda, A. Peitragalla, G. Scambia, Evaluation of pemetrexed (Alimta, LY231514) as second-line chemotherapy in persistent or recurrent carcinoma of the cervix: the CERVIX 1 study of the MITO (Multicentre Italian Trials in Ovarian Cancer and Gynecologic Malignancies) Group, Ann Oncol 21 (2010) 61-66.
[6] D. S. Miller, J. A. Blessing, D. C. Bodurka, A. J. Bonebrake, J. O. Schorge, Evaluation of pemetrexed (Alimta, LY231514) as second line chemotherapy in persistent or recurrent carcinoma of the cervix: a Phase II study of the Gynecologic Oncology Group, Gynecol Oncol 110 (2008) 65-70.
[7] A. M. Heeren, B. D. Koster, S. Samuels, D. M. Ferns, D. Chondronasiou, G. G. Kenter, E. S. Jordanova, T. D. de Gruijl, High and interrelated rates of PD-L1+CD14+antigen-presenting cells and regulatory T cells mark the microenvironment of metastatic lymph nodes from patients with cervical cancer, Cancer Immunol Res 3 (2015) 48-58.
[8] L. Deng, H. Liang, B. Burnette, M. Beckett, T. Darga, R. R. Weichselbaum, Y. X. Fu, Irradiation and anti-PD-L1 treatment synergistically promote antitumor immunity in mice, J Clin Invest 124 (2014) 687-695.
[9] K. P. Papadopoulos, M. L. Johnson, A. C. Lockhart, K. Moore, G. S. Falchook, S. C. Formenti, A. Naing, R. D. Carvajal, L. S. Rosen, G. J. Weiss, R. S. Leidner, J. Li, A. Paccaly, M. Feng, E. Stankevich, I. Lowy, M. G. Fury, M. R. Crittenden, First-in-human study of cemiplimab alone or in combination with radiotherapy and/or low-dose cyclophosphamide in patients with advanced malignancies, Clin Cancer Res 26 (2020) 1025-1033.
[10] R. W. Naumann, A. Hollebecque, T. Meyer, M.-J. Devlin, A. Oaknin, J. Kerger, J. M. Lopez-Picazo, J.-P. Machiels, J.-P. Delord, T. R. J. Evans, V. Boni, E. Calvo, S. L. Topalian, T. Chen, I. Soumaoro, B. Lin, J. Gu, R. Zwirtes, K. N. Moore, Safety and efficacy of nivolumab monotherapy in recurrent or metastatic cervical, vaginal, or vulvar carcinoma: results from the Phase I/II CheckMate 358 trial, J Clin Oncol 37 (2019) 2825-2834.
[11] A. D. Santin, W. Deng, M. Frumovitz, N. Buza, S. Bellone, W. Huh, S. Khleif, H. A. Lankes, E. S. Ratner, R. E. O'Cearbhaill, A. A. Jazaeri, M. Birrer, Phase II evaluation of nivolumab in the treatment of persistent or recurrent cervical cancer (NCT02257528/NRG-GY002), Gynecol Oncol 157 (2020) 161-166.
[12] M. Trivedi, B. Hoffner, J. Winkelmann, M. Abbott, O. Hamid, R. D. Carvajal, Programmed death 1 immune checkpoint inhibitors, Clin Adv Hematol Oncol 13 (2015) 858-868.
[13] R. L. Ferris, G. Blumenschein Jr, J. Fayette, J. Guigay, A. D. Colevas, L. Licitra, K. Harrington, S. Kasper, E. E. Vokes, C. Even, Nivolumab for recurrent squamous-cell carcinoma of the head and neck, N Engl J Med 375 (2016) 1856-1867.
[14] C. H. Lee, R. J. Motzer, Immune checkpoint therapy in renal cell carcinoma, Cancer J 22 (2016) 92-95.
[15] K. Chamoto, R. Hatae, T. Honjo, Current issues and perspectives in PD-1 blockade cancer immunotherapy, Int J Clin Oncol (2020).
[16] F. F. Gellrich, M. Schmitz, S. Beissert, F. Meier, Anti-PD-1 and novel combinations in the treatment of melanoma—an update, J Clin Med 9 (2020) 223.
[17] Y. K. Chae, A. Arya, W. lams, M. R. Cruz, S. Chandra, J. Choi, F. Giles, Current landscape and future of dual anti-CTLA4 and PD-1/PD-L1 blockade immunotherapy in cancer; lessons learned from clinical trials with melanoma and non-small cell lung cancer (NSCLC), J lmmunother Cancer 6 (2018) 39.
[18] J. S. Lee, E. Ruppin, Multiomics prediction of response rates to therapies to inhibit programmed cell death 1 and programmed cell death 1 ligand 1, JAMA Oncol 5 (2019) 1614-1618.
[19] A. Marabelle, M. G. Fakih, J. Lopez, M. Shah, R. Shapira-Frommer, K. Nakagawa, H. C. Chung, H. L. Kindler, J. A. Lopez-Martin, W. Miller, A. Italiano, S. Kao, S. A. Piha-Paul, J. P. Delord, R. R. McWilliams, D. Aurora-Garg, M. Chen, F. Jin, K. Norwood, Y. J. Bang, 1192O—Association of tumour mutational burden with outcomes in patients with select advanced solid tumours treated with pembrolizumab in KEYNOTE-158, Annals of Oncology 30 (2019) v477-v478.
[20] H. Kim, J.-H. Chung, PD-L1 testing in non-small cell lung cancer: past, present, and future, J Pathol Transl Med 53 (2019) 199-206.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A method of treating or inhibiting the growth of a tumor or improving overall survival of a cervical cancer patient, comprising:
(a) selecting a patient with cervical cancer; and
(b) administering to the patient a therapeutically effective amount of an antibody or antigen-binding fragment thereof that specifically binds programmed death 1 (PD-1), wherein the antibody or antigen-binding fragment thereof comprises three heavy chain complementarity determining regions (CDRs) (HCDR1, HCDR2 and HCDR3) of a heavy chain variable region (HCVR) comprising the amino acid sequence of SEQ ID NO: 1 and three light chain CDRs (LCDR1, LCDR2 and LCDR3) of a light chain variable region (LCVR) comprising the amino acid sequence of SEQ ID NO: 2.

2. The method according to claim 1, wherein the cervical cancer is selected from the group consisting of squamous cell carcinoma, adenocarcinoma and adenosquamous carcinoma.

3. The method according to claim 1, wherein the cervical cancer is advanced, recurrent, persistent and/or metastatic.

4. The method according to claim 1, wherein the cervical cancer is recurrent or metastatic.

5. The method according to claim 1, wherein the patient has received prior anti-cancer therapy, which was discontinued due to progression of disease and/or toxicity; or for whom the anti-cancer therapy was not appropriate.

6. The method according to claim 1, wherein the patient is resistant to, or the cervical cancer progressed after, prior treatment with an anti-cancer therapy.

7. The method according to claim 5, wherein the prior anti-cancer therapy comprises one or more of chemotherapy, surgery, radiation therapy, and/or anti-VEGF therapy.

8. The method according to claim 5, wherein the prior anti-cancer therapy comprises platinum-based chemotherapy.

9. The method according to claim 5, wherein the prior anti-cancer therapy comprises platinum-based chemotherapy selected from pemetrexed, topotecan, irinotecan, gemcitabine, and vinorelbine.

10. The method according to claim 1, wherein the patient has received prior chemotherapy or prior anti-VEGF therapy.

11. The method according to claim 10, wherein the prior anti-VEGF therapy comprises bevacizumab.

12. The method according to claim 1, wherein the patient has recurrent or metastatic cervical cancer with disease progression on or after chemotherapy.

13. The method according to claim 1, wherein the cervical cancer exhibits elevated expression of PD-L1.

14. The method according to claim 13, wherein the cervical cancer exhibits elevated expression of PD-L1 protein or PD-L1 mRNA.

15. (canceled)

16. The method according to claim 1, wherein the patient has tested positive for human papillomavirus (HPV).

17. The method according to claim 1, wherein the patient has tested negative for human papillomavirus (HPV).

18. The method according to claim 1, wherein the antibody or antigen-binding fragment thereof is administered as a monotherapy.

19. The method according to claim 1, wherein the administration of the antibody or antigen-binding fragment thereof promotes tumor regression, reduces tumor cell load, reduces tumor burden, and/or prevents tumor recurrence in the patient.

20. The method according to claim 1, wherein the administration of the antibody or antigen-binding fragment thereof leads to at least one improvement selected from increase in overall survival, progression free survival, overall response rate, complete response, partial response, and stable disease, as compared to patients treated with chemotherapy.

21. The method according to claim 20, wherein the administration of the antibody or antigen-binding fragment thereof leads to increased overall survival as compared to patients treated with chemotherapy.

22. The method according to claim 1, wherein the antibody or antigen-binding fragment thereof is administered in combination with a second therapeutic agent or therapy.

23. (canceled)

24. (canceled)

25. The method according to claim 1, wherein HCDR1 has comprises an amino acid sequence of SEQ ID NO: 3; HCDR2 comprises an amino acid sequence of SEQ ID NO: 4; HCDR3 comprises an amino acid sequence of SEQ ID NO: 5; LCDR1 comprises an amino acid sequence of SEQ ID NO: 6; LCDR2 comprises an amino acid sequence of SEQ ID NO: 7; and LCDR3 comprises an amino acid sequence of SEQ ID NO: 8.

26. The method according to claim 25, wherein the HCVR comprises an amino acid sequence of SEQ ID NO: 1 or the LCVR comprises an amino acid sequence of SEQ ID NO: 2.

27. (canceled)

28. The method according to claim 25, wherein the antibody or antigen-binding fragment thereof comprises an HCVR/LCVR amino acid sequence pair of SEQ ID NOs: 1/2.

29. The method according to claim 1, wherein the antibody comprises a heavy chain and a light chain, wherein the heavy chain has an amino acid sequence of SEQ ID NO: 9 or the light chain has an amino acid sequence of SEQ ID NO: 10.

30. (canceled)

31. The method according to claim 1, wherein the antibody comprises a heavy chain and a light chain, wherein the heavy chain has an amino acid sequence of SEQ ID NO: 9 and the light chain has an amino acid sequence of SEQ ID NO: 10.

32. The method according to claim 1, wherein the antibody or antigen-binding fragment thereof comprises a HCVR with 90%, 95%, 97%, or 98% sequence identity to SEQ ID NO: 1 or a LCVR with 90%, 95%, 97%, or 98% sequence identity to SEQ ID NO: 2.

33. (canceled)

34. The method according to claim 1, wherein the antibody or antigen-binding fragment thereof comprises a HCVR with 90%, 95%, 97%, or 98% sequence identity to SEQ ID NO: 1, and a LCVR with 90%, 95%, 97%, or 98% sequence identity to SEQ ID NO: 2.

35. The method according to claim 1, wherein the antibody is cemiplimab or a bioequivalent thereof.

36. (canceled)

37. (canceled)

38. The method according to claim 1, wherein the antibody or antigen-binding fragment thereof is administered at a dose of 5 mg to 1500 mg.

39. The method according to claim 1, wherein the antibody or antigen-binding fragment thereof is administered at a dose of 200 mg, 250 mg, 350 mg, 400 mg, 500 mg, 600 mg, 700 mg, 750 mg, 800 mg, 1000 mg, 1050 mg, or 1200 mg.

40. (canceled)

41. (canceled)

42. The method according to claim 38, wherein the antibody or antigen-binding fragment thereof is administered as one or more doses, wherein each dose is administered every week, two weeks, three weeks, four weeks, five weeks or six weeks.

43. The method according to claim 1, wherein the antibody or antigen-binding fragment thereof is administered intravenously or subcutaneously.

44. (canceled)

45. (canceled)

46. A kit comprising an antibody or antigen-binding fragment thereof that specifically binds programmed death 1 (PD-1) in combination with written instructions for use of a therapeutically effective amount of the antibody or antigen-binding fragment thereof for treating or inhibiting the growth of a tumor or improving overall survival of a patient with cervical cancer, wherein the antibody or antigen-binding fragment thereof comprises three heavy chain complementarity determining regions (CDRs) (HCDR1, HCDR2 and HCDR3) of a heavy chain variable region (HCVR) comprising the amino acid sequence of SEQ ID NO: 1 and three light chain CDRs (LCDR1, LCDR2 and LCDR3) of a light chain variable region (LCVR) comprising the amino acid sequence of SEQ ID NO: 2.

47. A method of treating a cervical cancer patient, comprising:
(a) selecting a patient with recurrent or metastatic cervical cancer with disease progression on or after chemotherapy; and
(b) administering cemiplimab or a bioequivalent thereof to the patient as a 350 mg intravenous infusion every 3 weeks,
thereby treating the cervical cancer patient.

48. The method according to claim 47, wherein the administration of the cemiplimab or bioequivalent thereof promotes tumor regression, reduces tumor cell load, reduces tumor burden, and/or prevents tumor recurrence in the patient.

49. The method according to claim 47, wherein the administration of the cemiplimab or bioequivalent thereof leads to at least one improvement selected from increase in overall survival, progression free survival, overall response rate, complete response, partial response, and stable disease, as compared to patients treated with chemotherapy.

50. A kit comprising cemiplimab or a bioequivalent thereof in combination with written instructions for use of 350 mg of cemiplimab every three weeks for treating or inhibiting the growth of a tumor or improving overall survival of a patient with cervical cancer, wherein the patient has recurrent or metastatic cervical cancer with disease progression on or after chemotherapy.


SEQUENCE LISTING (12 sequences)

SEQ ID NO: 1 - protein, 117 aa, Artificial sequence [sequence omitted]

SEQ ID NO: 2 - protein, 107 aa, Artificial sequence [sequence omitted]

SEQ ID NO: 3 - protein, 8 aa, Artificial sequence
  HCDR1
GFTFSNFG

SEQ ID NO: 4 - protein, 8 aa, Artificial sequence
  HCDR2
ISGGGRDT

SEQ ID NO: 5 - protein, 10 aa, Artificial sequence
  HCDR3
VKWGNIYFDY

SEQ ID NO: 6 - protein, 6 aa, Artificial sequence
  LCDR1
LSINTF

SEQ ID NO: 7 - protein, 3 aa, Artificial sequence
  LCDR2
AAS

SEQ ID NO: 8 - protein, 9 aa, Artificial sequence
  LCDR3
QQSSNTPFT

SEQ ID NO: 9 - protein, 444 aa, Artificial sequence
  HC 1-117; 118-444
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial sequence
  LC 1-108; 109-214
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 120 aa, Artificial sequence
  HCVR
EVQLVESGGGVVRPGGSLRLSCAASGFTFDDYGMTWVRQAPGRGLEWVSGIHWHGKRTGYADSVKGRFTISRDNAKKSLYLQMNSLKGEDTALYHCVRGGMSTGDWFDPWGQGTLVIVSS

SEQ ID NO: 12 - protein, 108 aa, Artificial sequence
  LCVR
DIQMTQSPSSLSASLGDRVTITCRASQSINSYLNWYQQKPGKAPKLLIYVASSLQSGVPSRFSGSGSGTEFTLTISNLQPEDFATYYCQQSYSTPPITFGQGTRLEIK